SEQUENCE LISTING (115 sequences)

SEQ ID NO: 1 - DNA, 131 bp, Neisseria gonorrhoeae
gcggttacaccacttttagtaccagaagaacagctaacgaaatgcgcaggctatagtcagtctttgcagtctataagctatattgttagtccggcagttgcagcactcttatactccgtttgggatttaaa

SEQ ID NO: 2 - DNA, 131 bp, Staphylococcus aureus; identical to SEQ ID NO: 1

SEQ ID NO: 3 - DNA, 131 bp, Enterococcus faecium
gcggttacgccacttttagtaccagaagaacagctgacgaaatgcgcaggctatagtcagtctttgcagtctataagctatattgttagtccggcggttgcagcactcttatactccgtttgggaactaaa

SEQ ID NO: 4 - DNA, 131 bp, Streptococcus pneumoniae
gcggttacgccacttttagtaccagaagaacagcttacgaaatgtgcaggctatagtcagtctttgcagtctataagctatattgttagtccggcggttgcagcactcttatactccgtttgggaactaaa

SEQ ID NO: 5 - DNA, 131 bp, Streptococcus salivarius
gcggttacgccacttttagtaccagaagaacagctaacgaaatgcgcaggctatagtcagtctttgcagtctataagctatattgttagtccggcagttgcagcactcttatactccgtttgggaactaaa

SEQ ID NO: 6 - DNA, 131 bp, Streptococcus pyogenes; identical to SEQ ID NO: 4

SEQ ID NO: 7 - DNA, 131 bp, Streptococcus pyogenes; identical to SEQ ID NO: 4

SEQ ID NO: 8 - DNA, 35 bp, Artificial Sequence
  Synthetic oligonucleotide
gcggttacgccacttttagtaccagaagaacagct

SEQ ID NO: 9 - DNA, 35 bp, Artificial Sequence
  Synthetic Oligonucleotide
tttagttcccaaacggagtataagagtgctgcaac

SEQ ID NO: 10 - DNA, 49 bp, Artificial Sequence
  Synthetic Oligonucleotide
  modified_base (33)..(33): abasic
  misc_feature (33)..(33): n is a, c, g, or t
caggctatagtcagtctttgcagtctataagcnatattgttagtccggc

SEQ ID NO: 11 - DNA, 20 bp, Artificial Sequence
  Synthetic Oligonucleotide
agagtttgatcctggctcag

SEQ ID NO: 12 - DNA, 19 bp, Artificial Sequence
  Synthetic Oligonucleotide
tgctgcctcccgtaggagt

SEQ ID NO: 13 - DNA, 30 bp, Artificial Sequence
  Synthetic Oligonucleotide
attcaccaagatattctacagtttcaattc

SEQ ID NO: 14 - DNA, 31 bp, Artificial Sequence
  Synthetic Oligonucleotide
ttgaattagacagtcatctattcaacttatc

SEQ ID NO: 15 - DNA, 30 bp, Artificial Sequence
  Synthetic Oligonucleotide
cactgtttacttttggtttaggatgaaagc

SEQ ID NO: 16 - DNA, 30 bp, Artificial Sequence
  Synthetic Oligonucleotide
ccaatatttatctggaacatctgtggtatg

SEQ ID NO: 17 - DNA, 31 bp, Artificial Sequence
  Synthetic Oligonucleotide
atggagttgaaagatttcttgattcctcaag

SEQ ID NO: 18 - DNA, 31 bp, Artificial Sequence
  Synthetic Oligonucleotide
caattagtatggacgatttagaagaaagagg

SEQ ID NO: 19 - DNA, 31 bp, Artificial Sequence
  Synthetic Oligonucleotide
acatatcaccaaactctgctaaatcttcaag

SEQ ID NO: 20 - DNA, 30 bp, Artificial Sequence
  Synthetic Oligonucleotide
tccaatttttcatgagctttgacatctccc

SEQ ID NO: 21 - DNA, 31 bp, Artificial Sequence
  Synthetic Oligonucleotide
tgagctaggatcggttagtaagacgtttaac

SEQ ID NO: 22 - DNA, 31 bp, Artificial Sequence
  Synthetic Oligonucleotide
tattatttcacctggggtaaagccgatatcg

SEQ ID NO: 23 - DNA, 30 bp, Artificial Sequence
  Synthetic Oligonucleotide
atgcagtaatgcggctttatccctaacgtc

SEQ ID NO: 24 - DNA, 30 bp, Artificial Sequence
  Synthetic Oligonucleotide
gtctggtcattgcctcttcgtaactcattc

SEQ ID NO: 25 - DNA, 30 bp, Artificial Sequence
  Synthetic Oligonucleotide
gacgtacagcaaaaacttgccgaattagag

SEQ ID NO: 26 - DNA, 30 bp, Artificial Sequence
  Synthetic Oligonucleotide
aggcagactgggtgtggcattgattaacac

SEQ ID NO: 27 - DNA, 30 bp, Artificial Sequence
  Synthetic Oligonucleotide
tcgctgatttaacagattcggttcgctttc

SEQ ID NO: 28 - DNA, 30 bp, Artificial Sequence
  Synthetic Oligonucleotide
ccgcaatcggattatagttaacaaggtcag

SEQ ID NO: 29 - DNA, 30 bp, Artificial Sequence
  Synthetic Oligonucleotide
gataccggctcaggcgcaactgtaagttac

SEQ ID NO: 30 - DNA, 30 bp, Artificial Sequence
  Synthetic Oligonucleotide
tcgctaaactcgaacaggactttggcggct

SEQ ID NO: 31 - DNA, 30 bp, Artificial Sequence
  Synthetic Oligonucleotide
catccgttacggcaaaaatgcgctggttcc

SEQ ID NO: 32 - DNA, 30 bp, Artificial Sequence
  Synthetic Oligonucleotide
caaattggcggcggcgttatcactgtattg

SEQ ID NO: 33 - DNA, 30 bp, Artificial Sequence
  Synthetic Oligonucleotide
gatcccaacggtgatattgtcactggtgtg

SEQ ID NO: 34 - DNA, 30 bp, Artificial Sequence
  Synthetic Oligonucleotide
tagtgctcagtgtcggcatcaccgagattg

SEQ ID NO: 35 - DNA, 30 bp, Artificial Sequence
  Synthetic Oligonucleotide
cgacttatgccaatgcgttgtcgaaccagc

SEQ ID NO: 36 - DNA, 30 bp, Artificial Sequence
  Synthetic Oligonucleotide
cagatcctcaactggatcaagcaggagatc

SEQ ID NO: 37 - DNA, 30 bp, Artificial Sequence
  Synthetic Oligonucleotide
gaatggagatctggaacagcaatcatacac

SEQ ID NO: 38 - DNA, 30 bp, Artificial Sequence
  Synthetic Oligonucleotide
tcgcattatcacttatggcatttgatgcgg

SEQ ID NO: 39 - DNA, 30 bp, Artificial Sequence
  Synthetic Oligonucleotide
ccatgcttctgttaatccgttgtttctttc

SEQ ID NO: 40 - DNA, 32 bp, Artificial Sequence
  Synthetic Oligonucleotide
ccagagaagtcttgatttccataatcaaaatc

SEQ ID NO: 41 - DNA, 30 bp, Artificial Sequence
  Synthetic Oligonucleotide
gacagatacaatgaatttggagcgtcaaag

SEQ ID NO: 42 - DNA, 30 bp, Artificial Sequence
  Synthetic Oligonucleotide
gtttattgtataccagtggtgcaattgcag

SEQ ID NO: 43 - DNA, 31 bp, Artificial Sequence
  Synthetic Oligonucleotide
cattatctgtagtgcatgaaacagtatacgg

SEQ ID NO: 44 - DNA, 31 bp, Artificial Sequence
  Synthetic Oligonucleotide
ccatcctttgcagaaactaataatactgctc

SEQ ID NO: 45 - DNA, 30 bp, Artificial Sequence
  Synthetic Oligonucleotide
cgaattggactacgcaattgaatcggcaag

SEQ ID NO: 46 - DNA, 30 bp, Artificial Sequence
  Synthetic Oligonucleotide
ttaattgagcaggctgtttcgggctgtgag

SEQ ID NO: 47 - DNA, 30 bp, Artificial Sequence
  Synthetic Oligonucleotide
gtaaaaacatatccacacgggctagacctc

SEQ ID NO: 48 - DNA, 30 bp, Artificial Sequence
  Synthetic Oligonucleotide
ctgcgggaacggttataactgcgttttcag

SEQ ID NO: 49 - DNA, 30 bp, Artificial Sequence
  Synthetic Oligonucleotide
cgcttctacgatattacgtggattctacga

SEQ ID NO: 50 - DNA, 30 bp, Artificial Sequence
  Synthetic Oligonucleotide
gtgcactgttgcaagaaaagtatcatgtgg

SEQ ID NO: 51 - DNA, 31 bp, Artificial Sequence
  Synthetic Oligonucleotide
gattgatttaagtatccaagagaaaccgagc

SEQ ID NO: 52 - DNA, 32 bp, Artificial Sequence
  Synthetic Oligonucleotide
cagggctatagtataagccatacttaaaacag

SEQ ID NO: 53 - DNA, 30 bp, Artificial Sequence
  Synthetic Oligonucleotide
ggtcaaatactacagatgcaacttaaacgg

SEQ ID NO: 54 - DNA, 31 bp, Artificial Sequence
  Synthetic Oligonucleotide
gctttttacttttccattgctgtaaccactc

SEQ ID NO: 55 - DNA, 30 bp, Artificial Sequence
  Synthetic Oligonucleotide
tcggtcttcttttcctctatctgcgcattg

SEQ ID NO: 56 - DNA, 32 bp, Artificial Sequence
  Synthetic Oligonucleotide
gcaatttctttcaaacgttctgcacgtaatag

SEQ ID NO: 57 - DNA, 31 bp, Artificial Sequence
  Synthetic Oligonucleotide
cattgctaattgtattccatgttttcgatgc

SEQ ID NO: 58 - DNA, 32 bp, Artificial Sequence
  Synthetic Oligonucleotide
ggcgttagagcaatggtttataaaaactatac

SEQ ID NO: 59 - DNA, 30 bp, Artificial Sequence
  Synthetic Oligonucleotide
gaataaagctgactaaacctgataatgcag

SEQ ID NO: 60 - DNA, 30 bp, Artificial Sequence
  Synthetic Oligonucleotide
ccggtacaaaatagtacgcaaaacgatata

SEQ ID NO: 61 - DNA, 30 bp, Artificial Sequence
  Synthetic Oligonucleotide
gataaaatcagccaaacctatcccatcgcg

SEQ ID NO: 62 - DNA, 30 bp, Artificial Sequence
  Synthetic Oligonucleotide
cgctatgtgctaaagcctgtgttgattttg

SEQ ID NO: 63 - DNA, 30 bp, Artificial Sequence
  Synthetic Oligonucleotide
cgcatgataaacgctgcgtttaatagatcc

SEQ ID NO: 64 - DNA, 30 bp, Artificial Sequence
  Synthetic Oligonucleotide
ccttaacaaaagccacaagcaaacttggta

SEQ ID NO: 65 - DNA, 30 bp, Artificial Sequence
  Synthetic Oligonucleotide
gcttttatcagtgtgacttcagccactgtc

SEQ ID NO: 66 - DNA, 30 bp, Artificial Sequence
  Synthetic Oligonucleotide
cagcaagaggatttctcacgccaggatttg

SEQ ID NO: 67 - DNA, 30 bp, Artificial Sequence
  Synthetic Oligonucleotide
cattgctccagttgttttcaaacagctcgc

SEQ ID NO: 68 - DNA, 30 bp, Artificial Sequence
  Synthetic Oligonucleotide
cagaagtacatcttatggctgacttgattg

SEQ ID NO: 69 - DNA, 30 bp, Artificial Sequence
  Synthetic Oligonucleotide
gtgatcaccgagttgccgtatcaggtcaac

SEQ ID NO: 70 - DNA, 30 bp, Artificial Sequence
  Synthetic Oligonucleotide
caagctggccggcatttccaacattgagga

SEQ ID NO: 71 - DNA, 30 bp, Artificial Sequence
  Synthetic Oligonucleotide
ggtcaacgtaatagcggatcagctggtcca

SEQ ID NO: 72 - DNA, 31 bp, Artificial Sequence
  Synthetic Oligonucleotide
gtctgcagctgggtgtgcttgtaaaggttat

SEQ ID NO: 73 - DNA, 30 bp, Artificial Sequence
  Synthetic Oligonucleotide
gtgtgaagggcttcgtcgagtacatcaaca

SEQ ID NO: 74 - DNA, 30 bp, Artificial Sequence
  Synthetic Oligonucleotide
cacggtcgaatatcactacgacatcctcgc

SEQ ID NO: 75 - DNA, 30 bp, Artificial Sequence
  Synthetic Oligonucleotide
gatgtacttgttgatgacgcgcgtcatcgc

SEQ ID NO: 76 - DNA, 30 bp, Artificial Sequence
  Synthetic Oligonucleotide
cagaagaaaccagcttgtccttcgtctgcg

SEQ ID NO: 77 - DNA, 30 bp, Artificial Sequence
  Synthetic Oligonucleotide
gttgaaatcactcgcgatggtgcaatctac

SEQ ID NO: 78 - DNA, 30 bp, Artificial Sequence
  Synthetic Oligonucleotide
gtacagcacccaagtctaaaacaggtacca

SEQ ID NO: 79 - DNA, 30 bp, Artificial Sequence
  Synthetic Oligonucleotide
cttccacttggaaaccattatcttctcctt

SEQ ID NO: 80 - DNA, 30 bp, Artificial Sequence
  Synthetic Oligonucleotide
ggtctccttgtcttcgttaagataagaaac

SEQ ID NO: 81 - DNA, 30 bp, Artificial Sequence
  Synthetic Oligonucleotide
ctatctatgatgccatggttcgtatgtcac

SEQ ID NO: 82 - DNA, 30 bp, Artificial Sequence
  Synthetic Oligonucleotide
ggtaacatcatggggaatttccacccacac

SEQ ID NO: 83 - DNA, 30 bp, Artificial Sequence
  Synthetic Oligonucleotide
gcaatctcagacaaacgcgcctcagtataa

SEQ ID NO: 84 - DNA, 30 bp, Artificial Sequence
  Synthetic Oligonucleotide
cagtcgaaccattgaccaagaggtttggaa

SEQ ID NO: 85 - DNA, 30 bp, Artificial Sequence
  Synthetic Oligonucleotide
gtacttcgacgagaccattgacaagtccac

SEQ ID NO: 86 - DNA, 30 bp, Artificial Sequence
  Synthetic Oligonucleotide
gatgatgaccgagaagggcacgttcatcat

SEQ ID NO: 87 - DNA, 30 bp, Artificial Sequence
  Synthetic Oligonucleotide
gaacaagttttccaacagcgtctgcgctga

SEQ ID NO: 88 - DNA, 30 bp, Artificial Sequence
  Synthetic Oligonucleotide
cagcccgagcttcttgttgaccttatagcg

SEQ ID NO: 89 - DNA, 30 bp, Artificial Sequence
  Synthetic Oligonucleotide
gaagtatcaaagaatgagagagtagaaacg

SEQ ID NO: 90 - DNA, 30 bp, Artificial Sequence
  Synthetic Oligonucleotide
ggatatgaaggttcttccaaaattacttag

SEQ ID NO: 91 - DNA, 30 bp, Artificial Sequence
  Synthetic Oligonucleotide
agagcttcacccattcccataaaagaaatg

SEQ ID NO: 92 - DNA, 31 bp, Artificial Sequence
  Synthetic Oligonucleotide
ggaagtataaaacttgatctgttatctcatc

SEQ ID NO: 93 - DNA, 35 bp, Artificial Sequence; identical to SEQ ID NO: 8
  Synthetic Oligonucleotide

SEQ ID NO: 94 - DNA, 35 bp, Artificial Sequence; identical to SEQ ID NO: 9
  Synthetic Oligonucleotide

SEQ ID NO: 95 - DNA, 47 bp, Artificial Sequence
  Synthetic Oligonucleotide
  modified_base (32)..(32): abasic
  misc_feature (32)..(32): n is a, c, g, or t
taccttggatattcaccgaacactagggttgntcttgcacactcaag

SEQ ID NO: 96 - DNA, 46 bp, Artificial Sequence
  Synthetic Oligonucleotide
  modified_base (31)..(31): abasic
  misc_feature (31)..(31): n is a, c, g, or t
tttaaagatagtggtatgcttagttttcgantgactccacgcaagg

SEQ ID NO: 97 - DNA, 46 bp, Artificial Sequence
  Synthetic Oligonucleotide
  modified_base (31)..(31): abasic
  misc_feature (31)..(31): n is a, c, g, or t
tggccagaactgacaggcaaacagtggcagngtatccgcctgctgc

SEQ ID NO: 98 - DNA, 46 bp, Artificial Sequence
  Synthetic Oligonucleotide
  modified_base (31)..(31): abasic
  misc_feature (31)..(31): n is a, c, g, or t
ttcgcaaatactttatcgtgctgatgagcgntttgcgatgtgcagc

SEQ ID NO: 99 - DNA, 46 bp, Artificial Sequence
  Synthetic Oligonucleotide
  modified_base (31)..(31): abasic
  misc_feature (31)..(31): n is a, c, g, or t
tgctgccgctgtgctggctcgcagccagcancaggccggcttgctg

SEQ ID NO: 100 - DNA, 46 bp, Artificial Sequence
  Synthetic Oligonucleotide
  modified_base (31)..(31): abasic
  misc_feature (31)..(31): n is a, c, g, or t
ttgctggttcgacccagccattggcggcganagtcaggctgtgttg

SEQ ID NO: 101 - DNA, 46 bp, Artificial Sequence
  Synthetic Oligonucleotide
  modified_base (31)..(31): abasic
  misc_feature (31)..(31): n is a, c, g, or t
ttgggtttcgcaagaaataacccaaaaaatnggattaaataaaatc

SEQ ID NO: 102 - DNA, 46 bp, Artificial Sequence
  Synthetic Oligonucleotide
  modified_base (31)..(31): abasic
  misc_feature (31)..(31): n is a, c, g, or t
tgggaggatgtaaaagtcaacattatagatncgccaggccatatgg

SEQ ID NO: 103 - DNA, 46 bp, Artificial Sequence
  Synthetic Oligonucleotide
  modified_base (31)..(31): abasic
  misc_feature (31)..(31): n is a, c, g, or t
tcaggctgcagtacggaatctttcgtattcntcaggaagtcgagcc

SEQ ID NO: 104 - DNA, 46 bp, Artificial Sequence
  Synthetic Oligonucleotide
  modified_base (31)..(31): abasic
  misc_feature (31)..(31): n is a, c, g, or t
tgccgccaaatcctttctgggcttccattgntttatctgtatcacc

SEQ ID NO: 105 - DNA, 46 bp, Artificial Sequence
  Synthetic Oligonucleotide
  modified_base (31)..(31): abasic
  misc_feature (31)..(31): n is a, c, g, or t
tattatttttatgcagaccaagctcttgcangtgcagaatgatttg

SEQ ID NO: 106 - DNA, 46 bp, Artificial Sequence
  Synthetic Oligonucleotide
  modified_base (31)..(31): abasic
  misc_feature (31)..(31): n is a, c, g, or t
tgtgttgaatggttagcagctgcagttgtantatatttctgtggtg

SEQ ID NO: 107 - DNA, 46 bp, Artificial Sequence
  Synthetic Oligonucleotide
  modified_base (31)..(31): abasic
  misc_feature (31)..(31): n is a, c, g, or t
tattttactgacacttatggcacggtctatnatacgaccatgctcc

SEQ ID NO: 108 - DNA, 46 bp, Artificial Sequence
  Synthetic Oligonucleotide
  modified_base (31)..(31): abasic
  misc_feature (31)..(31): n is a, c, g, or t
ttcagctatgccgatctgcgcgatgccagtntcaaggcctgccgtc

SEQ ID NO: 109 - DNA, 46 bp, Artificial Sequence
  Synthetic Oligonucleotide
  modified_base (31)..(31): abasic
  misc_feature (31)..(31): n is a, c, g, or t
tctagcgatcgggtcggtttacgcatcgtcntcgagatcaagcgcg

SEQ ID NO: 110 - DNA, 47 bp, Artificial Sequence
  Synthetic Oligonucleotide
  modified_base (31)..(31): abasic
  misc_feature (31)..(31): n is a, c, g, or t
tgcagtggaacgacagctacaacgagaacgngctgtgcttcaccaac

SEQ ID NO: 111 - DNA, 46 bp, Artificial Sequence
  Synthetic Oligonucleotide
  modified_base (31)..(31): abasic
  misc_feature (31)..(31): n is a, c, g, or t
tcttgaaaaatgtgaccttgtccttgacggncaagcgaacagatga

SEQ ID NO: 112 - DNA, 46 bp, Artificial Sequence
  Synthetic Oligonucleotide
  modified_base (31)..(31): abasic
  misc_feature (31)..(31): n is a, c, g, or t
tgaaatgcacggtaataacggttctatgganggagatcctcctgcg

SEQ ID NO: 113 - DNA, 47 bp, Artificial Sequence
  Synthetic Oligonucleotide
  modified_base (31)..(31): abasic
  misc_feature (31)..(31): n is a, c, g, or t
tgcgcatcgaccgcaaacgccggcaaccggncaccgtgctgctcaag

SEQ ID NO: 114 - DNA, 46 bp, Artificial Sequence
  Synthetic Oligonucleotide
  modified_base (31)..(31): abasic
  misc_feature (31)..(31): n is a, c, g, or t
tcatcacaatgcggatgtaattttgggtgtnaattttgtgctacag

SEQ ID NO: 115 - DNA, 49 bp, Artificial Sequence; identical to SEQ ID NO: 10
  Synthetic Oligonucleotide
  modified_base (33)..(33): abasic
  misc_feature (33)..(33): n is a, c, g, or t

(19) United States
(12) Patent Application Publication
(10) Pub. No.: US 2020/0087709 A1
(43) Pub. Date: Mar. 19, 2020

(54) DIRECT-TO-CONSUMER GENOMIC DIAGNOSTIC DEVICE

(71) Applicant: MicroInvestigate, LLC, Arlington, VA (US)

(72) Inventors: John BRACHT, Washington, DC (US); Megan NELSON, Arlington, VA (US); William BELLOWS, Lincoln, VA (US); Kathryn WALTERS-CONTE, Silver Spring, MD (US)

(21) Appl. No.: 16/352,083

(22) Filed: Mar. 13, 2019

Related U.S. Application Data
(60) Provisional application No. 62/642,107, filed on Mar. 13, 2018.

(51) Int. Cl. C12Q 1/689; C12Q 1/6844

(52) U.S. Cl. CPC C12Q 1/689; C12Q 2600/158; C12Q 2600/16; C12Q 1/6844

(57) ABSTRACT

A point-of-care diagnostic system comprising a method and related device is provided for detecting and/or quantitating pathogens and/or antimicrobial resistance in a sample, such as a bodily fluid. In various embodiments, the described method comprises Recombinase Polymerase Assay (RPA) using specifically designed primer sets and probes. In various embodiments, the described system comprises a detection module, and a data analysis and processing module, where the detection module comprises a fluid handling system for performing Recombinase Polymerase Assay (RPA) on the sample.

### BACKGROUND

Today, receiving a diagnosis of a common illness requires time consuming doctor's visits or time-spent waiting for results. Unfortunately, primary care physicians and providers (PCPs) have a few options for diagnosing these common illnesses and determining whether or not there is need for antibiotics. Most common methods used are a visual diagnosis, individual (and often unreliable) specialized-rapid tests, time-consuming outsourcing to labs. Moreover, from discussions with doctors, one of the largest struggles for PCPs is working with insurance companies for billing for tests and exams. While more advanced tools may be accessible at hospitals, due to the larger budgets, PCPs are typically the first step in diagnosis. A study conducted in 2016, at least one in every 20 adults who seeks medical care in a U.S. emergency room or community health clinic may walk away with the wrong diagnosis with total estimates of approximately 12 million Americans a year could be affected by such errors. Of those misdiagnosis mistakes, about 6 million could potentially result harm.

Infectious diseases are one of the leading causes of human mortality worldwide and require accurate diagnostic methods to optimize clinical management of infected patients. Misdiagnoses of causative infectious agents is a pervasive problem in healthcare resulting in rampant over-prescription of antibiotics leading to the emergence of multi-drug resistance bacterial strains. An NSF funded study demonstrated a marked tendency for doctors to simplify diagnosis based on symptom lists and not tests, commonly leading to the prescribing of antibiotics as a situational default when test results are inconclusive. An ideal detection assay is sensitive, specific, and rapid for maximal patient recovery with minimal clinical complications. Previously, the long-standing gold standard for diagnosis was culture in growth-supporting media, which included a minimum 24 h protocol consisting of isolation, identification, and antibiotic susceptibility testing. The introduction of polymerase chain reaction (PCR) in the 1980s resulted in the development of a multitude of diagnostic tools which improved the efficiency and characterization of infectious diseases through DNA identification. However, PCR-based testing lacked the sensitivity and specificity for discrimination among bacterial species. Real-time PCR (RT-PCR) targeting of shorter fragments using fluorescent probes greatly improved detection speed, sensitivity, and specificity, however often requires multiple days to yield results. The availability of next generation sequencing (NGS) together with decreasing costs for sequences and reagents has revolutionized the field of infectious disease diagnosis with more than 38,000 bacterial and 5,000 viral genomes sequenced to date, including representatives of all significant pathogens. Results can be obtained within a few days for less than $500. Overall, NGS has enabled understanding of microorganism pathogenesis/evolution and improved diagnostic tools including assays for detection of virulence factors and antibiotic resistance determinants. However, several challenges remain including rapid access of clinical microbiology laboratories to sequencing platforms and standardized, fully automated sequence interpretation independent of sequencing platform and microorganism species.

Molecular approaches significantly improve diagnostics relative to slow, culture-based methods or rapid point-of-care tests that have low sensitivity. In multiple studies, qPCR method sensitivity and specificity are generally found to be in the 95-100% range. In a direct comparison of qPCR to culture-based identification, the molecular approach was between 7 and 219% more sensitive for Group A streptococci, Legionella spp., Vancomycin-resistant enterococci, Vaicella-zoster virus (chicken pox), Herpes Simplex Virus, and Cytomegalovirus while shortening turnaround time from 1-14 days to 30-45 minutes.

Multiplexing PCR assays have enabled simultaneous detection and discrimination of various microorganisms. To date, several genome-based PCR tests have multiplexed detection of the various pathogens potentially involved in a given infectious syndrome are commercially available. These include the LightCycler SeptiFast (Roche, Mannheim, Germany) and GeneXpert (Cepheid, Sunnyvale, Calif., USA). Microarrays, for example, have enabled the detection of more than 2,000 viral and 900 bacterial species simultaneously. They can be automated and are a fast, sensitive, high throughput genotyping tool. Although highly discriminatory, microarray-based methods cannot identify genetic fragments for which no probe is used.

In addition to the development of highly specific PCR assays, the study of genomic sequences has optimized sensitivity of detection either by selecting a gene or fragment of noncoding DNA present as several copies in the genome or by designing nested PCR assays targeting previously unused genomic fragments. Molecular typing (fingerprinting) methods are classified as either non-sequence-based or sequence-based genotyping depending on their design. Non-sequence-based genotyping methods include pulsed-field gel electrophoresis (PFGE), PCR-restriction fragment length polymorphism (PCR-RFLP), multiple-locus variable-number tandem repeat analysis (MLVA), single-nucleotide polymorphisms (SNPs) and microarrays.

PFGE and PCR-RFLP have long been considered as ‘gold standard’ genotyping methods. These methods are DNA-banding-pattern-based methods that compare the electrophoretic profiles of restriction-enzyme cut genomes or PCR-amplified genes from various strains. Initially these methods relied on uncharacterized genomic differences, however the use of genomic sequences markedly improves the sensitivity and specificity of PFGE and PCR-RFLP by enabling in silico prediction of the most appropriate restriction profiles of rare-cutter enzymes for a given bacterium. In an alternative approach, Lang and colleagues used genomics to design Pan-PCR software dedicated to the identification of the presence/absence of strain-specific PCR targets. Although this method is rapid, easy to perform, and requires only a thermocycler, it may not be adapted to species with highly conserved genomes (not varying among various strains).

MLVA is another non-sequence-based genotyping method which utilizes the number and length of variable tandem repeats (VNTRs) present in a genome and is applicable to a variety of pathogens. It is a rapid, easy to perform, affordable, and reproducible genotyping method with high discriminatory power. Currently, MLVA is a reference genotyping method for many bacteria and has been used to investigate infection outbreak, however, is non-adaptable for some species of bacteria lacking tandem repeats.

SNPs are another widely used typing method which has been improved through the use of genomic sequences. This method is based on point-nucleotide changes between strains of a given species and has enabled genotyping of several bacterial pathogens. In comparison to other genotyping methods, SNP based methods are rapid, sensitive, and easy to perform with unambiguous result interpretation. However, interpretation is highly dependent on the algorithm, reference sequence, and sequencing platform used highlighting the need for method standardization.

In comparison to non-sequence-based methods, sequence-based genotyping has a major advantage of being highly reproducible, since the sequence fragments on which it is based are stored in public databases. It relies on the selection of several genomic targets or the whole genomic sequence. Single locus sequence typing methods require in silico identification of a highly variable gene. MLST is one of the most frequently used sequence-based genotyping methods and is based on the combination of genotypes from several individual genes (generally housekeeping genes) for bacterial strain characterization. MLST is useful for characterizing pathogens with highly variable genomes among strains, however have less discriminating power among bacterial strains with highly conserved genomes. This method is highly valuable when implemented with the BIGSdb platform which enables data standardization. In a similar fashion, multispacer typing (MST) is based on the assumption that intergenic spacers have greater variability compared to genes and combines sequences from the most variable intergenic spacers between aligned genomes of bacterial strains. However, whole genome sequencing (WGS) using NGS is the ultimate discriminatory sequencing-based genotyping method and is commonly used for epidemiological investigations to evaluate global disease transmission. Overall, NGS has the potential to change clinical microbiology in number of ways. First, the increasing number of genome sequences will enable the development of new and improved pathogen specific or syndrome-based single or multiplexed RT-PCR assays and will aid in the refinement of DNA targets, primers, and probes used in existing tests. Secondly, the increase in speed, decreasing costs, and discriminatory power of NGS make it an ideal tool for routine use in diagnostic microbiology laboratories. Thirdly, it has the potential to replace existing tests for identification of antibiotic-resistance mechanisms and virulence determinants.

TwistDx has developed Recombinase Polymerase Amplification (RPA), an isothermal nucleic amplification technology that is a hugely versatile alternative to PCR for fast, portable, nucleic acid detection assays. RPA is inherently applicable to applications such as infectious disease diagnostics and is ideally suited to field, point-of-care, and other settings with minimal resources. It is fast, transportable, user friendly, and highly sensitive with results generated within 3-10 minutes. Unlike PCR, RPA does not require thermocycling or chemical melting negating the need for an expensive thermocycler or any additional equipment of reagents. The reaction works optimally at a temperature of around 37-42° C., which is lower than for other isothermal approaches, however, will also work over a wide range of ambient temperatures. The dry-formulated reagents exhibit excellent stability at ambient temperature for over 12 months and the lyophilized reagent pellet involves a simple workflow that can be carried out without specialist training. RPA can be used to replace PCR in a wide variety of applications and end users can design personalized ultrasensitive assays using their own primers. RPA can also be adapted to a range of microfluidic, lateral flow, and other devices, and by adding reverse transcriptase to the reaction mix to amplify and detect RNA.

### Application of RPA to Genomic Detection of Antimicrobial Resistance Elements.

Combating antimicrobial resistance (AMR) is a national and international priority. The U.S. National Institutes of Health [1], Center for Disease Control [2], World Health Organization [3], and United Nations [4] have prioritized the issue. On Sep. 18, 2014 former President Barack Obama issued AMR-focused Executive Order 13676 [5], which was followed by a National Action Plan for Combating Antibiotic Resistant Bacteria [6].

However, surveillance of antimicrobial resistance is a significant challenge [3, 6, 7], causing difficulties in obtaining a realistic threat measurement [3, 6], and impairing the ability to form future projections [8]. Current methods of assessing antimicrobial resistance are extremely slow, requiring days to weeks of culture time, and are also costly in terms of laboratory materials and technician effort. Correspondingly, they are deployed unevenly, biasing our estimates of AMR worldwide and inhibiting our ability to accurately assess this threat to human health [8]. Responding to calls for new diagnostic methods to address this unmet need [7], described herein is a simple, rapid, culture-free genomic method for detecting antimicrobial resistance within 10 minutes of assay time. Also provided is a simple raw-lysate preparation method that does not require nucleic acid purification. Together these innovations address a critical need in surveillance of antimicrobial resistance.

Recombinase Polymerase Amplification (RPA), a relative of Polymerase Chain Reaction (PCR), uses Recombinase-primer complexes to identify and denature the genomic segment of interest, along with single-stranded DNA-binding proteins to stabilize the open DNA [9]. Detection is similar to Taq-Man hydrolysis probes [10] except that the probe contains an internal abasic site analog, such as tetrahydrofuran, that is cleaved by Endonuclease IV (nfo) [11] during the course of amplification [9]. In one embodiment, the polymerase used is strand-displacing Bsu [9], which is more resistant to chemical inhibition than Taq, rendering RPA more robust than PCR [12]. Because proteins rather than heat perform DNA denaturation, RPA occurs isothermally, usually in a range of about 37° C.-42° C., and multiple reports document improved speed for RPA relative to PCR, often with detection within 5-7 minutes [12-14]. In addition, RPA demonstrates extreme sensitivity, often detecting tens of copies of a nucleic acid target [9, 13-16]. While RPA has not been widely implemented in clinical settings, it has been proven capable of detecting bacterial, viral, and protozoan human pathogens. Eukaryotic pathogens detected with RPA include the blood-fluke Schistosomajaponicum [14], the diarrheal protozoan pathogens Giardia, Cryptosporidium, and Entamoeba [16], and Cryptosporidium species [17]. Viral pathogens detected by RPA include HIV [18, 19], Chikungunya virus (CHIKV) [13], Rift Valley Fever virus [20, 21], Middle East respiratory syndrome coronavirus [22], foot-and-mouth disease virus (FMDV) [23], Bovine Coronavirus [24], and Crimean-Congo Haemorrhagic fever Virus (CCHFV) [25]. Bacterial pathogens detected by RPA include Mycoplasma tuberculosis [26, 27] Neisseria gonorrhoeae, Salmonella enterica and methicillin-resistant Staphylococcus aureus (MRSA)[28], Chlamydia trachomatis [29], Francisella tularensis [30], Group B Streptococci [31], Orientia tsutsugamushi (scrub typhus) and Rickettsia typhi (murine typhus)[15].

In diagnostic applications RPA has been shown to be highly specific and thus resistant to false positives (Type I errors). In several cases, 100% specificity was shown [13-15, 19]. Because of the health risks of erroneous detection and treatment, high specificity is an important characteristic of diagnostic assays. Type II errors (false negatives) are always possible if the pathogenic target is present at a low level in a sample, but the exquisite sensitivity of RPA minimizes this risk.

The diagnosis of pathogens by genomic methods has to date been driven by physician-initiated mail-in Quantitative Polymerase Chain Reaction (qPCR), but a newer, related, isothermal method, Recombinase Polymerase Assay (RPA) has shown significant improvements over PCR in terms of simplicity, speed, and robustness. Over 200 studies have demonstrated RPA's functionality, but an easy-to-use clinical implementation of the technology has not been accomplished. In one aspect, a fully automated robotic kiosk which incorporates RPA will be used for sample processing and RPA setup. This kiosk will detect both pathogens and antimicrobial resistance genes. This fully automated diagnostic system will allow ‘drop and go’ sample deposition, yielding a report which will be accessible by the patient through their healthcare portal and their physician.

Here, we developed and tested a novel RPA assay for the detection of the Macrolide Efflux A, or mef(A) gene, an efflux pump rendering host bacteria resistant to 14- and 15-membered macrolide antibiotics (including erythromycin A and azithromycin) [32, 33]. This gene can be found within Streptococcus pyogenes, the largest member of the Lancefield group A streptococci, where (if present) it is encoded on a transposon that is integrated into a prophage [34, 35]. While initially identified in S. pyogenes and S. pneumoniae [32] it has since been identified in an extremely wide range of gram-positive and negative bacteria worldwide [36] consistent with horizontal transfer of antimicrobial resistance genes.

Using purified DNA, a panel of bacteria cultures, and broth dilution antimicrobial resistance testing, extreme sensitivity and specificity of the RPA assay was demonstrated, and positive results correctly predict antimicrobial resistance was confirmed. The described RPA assay uncovered an unexpected occurrence of the mef(A) gene within commensal Streptococcus salivarius strain, and subsequent laboratory testing confirmed that this strain has genuine antimicrobial resistance. While S. salivarius has been known to frequently harbor antimicrobial resistance genes [37], this is the first case, to our knowledge, of antimicrobial resistance first discovered by RPA and confirmed by more traditional methods.

### BRIEF DESCRIPTION OF THE DRAWINGS

FIGS. 1A-C show design and sensitivity testing of Recombinase Polymerase Assay (RPA) against mef(A) gene. FIG. 1A shows a schematic of probe and primer design. Taq-Man-style hydrolysis probe is cleaved by nfo endonuclease during amplification, releasing the quencher and activating FAM signal. Quencher serves as 3′ blocking moiety. FIG. 1B shows RPA sensitivity testing using serial dilutions of DNA from mef(A)-positive Streptococcus pyogenes strain MGAS10394. FIG. 1C shows a comparison with qPCR using the primers from RPA (B), but using Sybr Green as readout instead of FAM (the probe was not used).

FIGS. 2A-C show bacterial panel for RPA assay and validation of raw lysate method. FIG. 2A shows a schematic of culture and bacterial lysate method. FIG. 2B shows Mef(A) RPA results for S. pyogenes, S. agalactiae, and S. salivarius. DNA concentration in raw lysates was measured and total amount of DNA loaded into each reaction is indicated. Lines are labeled with species name and whether they are known mef(A) positive (+) or negative (−); S. salivarius was isolated from a patient so was unknown prior to this study. FIG. 2C shows Mef(A) RPA results for S. pneumoniae and E. faecium. DNA concentration in raw lysates was measured and total amount of DNA loaded into each reaction is indicated. Lines are labeled with species name and whether they are known mef(A) positive (+) or negative (−).

FIGS. 3A-B show confirmation of mef(A) gene in Streptococcus salivarius by PCR and sequencing. FIG. 3A shows PCR against mef(A) was performed with the RPA primers (Table 1). 16S rDNA was amplified as a loading control with universal bacterial primers 27F and 388R (Table 1). FIG. 3B shows alignment of N. gonorrhoeae (SEQ ID NO: 1). S. aureus (SEQ ID NO: 2), E. faecium (SEQ ID NO: 3), S. pneumoniae (SEQ ID NO: 4), S. salivarius (SEQ ID NO: 5), MGAS10394 (SEQ ID NO: 6), and MGAS10394 reference CP000003.1 (SEQ ID NO: 7), and shows that S. pyogenes and S. salivarius mef(A) genes are different. MGAS10394 reference CP000003.1 is set as reference and differences are highlighted in figure. PCR-derived sequences are marked with an asterisk.

FIGS. 4A-J show antibiotic testing to confirm erythromycin resistance in S. salivarius, MGAS10394, and S. agalactiae. Ampicillin (always the second panel) serves as negative control (all strains susceptible). FIGS. 4A-B show media only. FIGS. 4C-D show MGAS10394 (mef(A) positive). FIGS. 4E-F show MGAS6180 (mef(A) negative). FIGS. 4G-H show S. agalactiae (ermB positive and mef(A) negative). FIGS. 4I-J show S. salivarius (mef(A) positive). Drug concentrations in all panels given in μg/ml.

FIG. 5 shows specificity testing using combined raw bacterial lysates and spiked-in purified human genomic DNA. H.s. DNA derived from human adipose-derived stem cells. Mixes positive for mef(A) are indicated with an asterisk and the mef(A)-containing lysates indicated in bold along with the total DNA in the mixture.

FIGS. 6A-D shows an overview of infectious disease diagnosis and empowerment of patients. FIG. 6A shows the current standard of care for respiratory infection. FIG. 6B shows the described knowledge-based care of respiratory infection. FIG. 6C shows the general diagnostic model for healthcare system. Notice patient does not obtain a communication of a diagnosis until after engaging the health-care system and penetrating the diagnostic firewall (jagged orange line). FIG. 6D shows revision of the diagnostic process. In this new flow, a described diagnostic device is available directly to a patient, providing rapid, e.g., within 1 hr, diagnostic information, which accelerates the diagnostic cycle, and improves outcomes.

FIG. 7 shows etiologic agents of human respiratory diseases to be targeted using the described method and device. Despite its non-respiratory nature, Zika is included due to its frequent lack of symptoms and known presence in saliva.

FIG. 8 shows MecA RPA assay using raw bacterial lysate (purple line marked with squares) or no-template control (NTC, green line marked with asterisks). Overnight MGAS10394 culture was boiled for 3 min at 95 degrees and diluted 100× in purified water, then used in assay. Time to threshold was 19.5 min.

FIG. 9 shows Panel design for MicroInvestigate system. The assays to be used are indicated as green or blue dots for agent identification and antibiotic resistance elements, respectively. The cladogram indicates relative phylogenetic relationships between bacterial and fungus outgroup (Candida spp.). Dotted lines connect plasmid-or-transposon encoded antibiotic resistance genes and the species reported to contain them. Genomic conservation allows the identification of primers at internal nodes for verification of specific organisms (branch tips assays) and enabling the high-level categorization of novel organisms even in absence of specific identifier.

FIG. 10 shows MicroInvestigate system flow. The sample will be collected at the MI Station. Next, the sample is analyzed and evaluated and the patient receives the result. The patient has the option to refer to a local doctor or have the results sent to a family practioner. The patient and the doctor then determine the next steps for treatment.

FIG. 11 shows how MicroInvestigate Disrupts Diagnostic Procedure. The left side depicts multiple appointments under the current flow of diagnostics and treatment. The right side depicts reversing the sequence of initial diagnostics and scheduling, to testing and diagnosing first before scheduling an appointment and followed by treatment.

FIGS. 12A-C show a design, according to one embodiment, of a customized kiosk. FIG. 12A shows a computer-aided design (CAD) of the three-axis of movement arm and table where the sample preparation and RPA reaction would occur. Heating block and electronic readout system indicated in red. FIG. 12B shows a close up of the pipette and robotic arm which will position objects and pipet liquids. FIG. 12C shows a semi-enclosed device. The computer and customer interface are not shown in the figure.

FIGS. 13A-C show reports generated by MiKi device. FIG. 13A shows the clear, concise response that will be presented to the patient. Patient will also receive the physician report. FIG. 13B shows a physician report, which will also be provided for patients to bring to physician's office when necessary. FIG. 13C shows a smartphone report conveying the bare minimum of actionable data.

### DETAILED DESCRIPTION

Misdiagnosis-errors in identification of causative infectious agents—is a pervasive problem in healthcare. As a consequence, the over-prescription of antibiotics is rampant, leading to the emergence of multi-drug resistant bacterial strains.

Through NSF funded research into this problem, a marked tendency of doctors to simply diagnose based on symptom lists, not tests—or to prescribe antibiotics as a situational default when tests results are inconclusive has been shown. Furthermore, the ongoing use of bacterial culture methods may be considered a historical anachronism, often requiring multiple days to yield results. Meanwhile available genomic (qPCR) assays are not utilized well, and still require doctors to send samples off to be tested by an outside laboratory, thereby negating the inherent speed these types of tests could provide. Meanwhile the few rapid (15 min or less) assays that have been approved for clinical use are relatively insensitive (often missing upwards of 30% of true cases) or may be too specific (identifying Strep A only, missing Strep B or C entirely). Genomic assays promise to provide fast, accurate molecular identification of pathogens, but as described above they are not being deployed in ways that benefit healthcare practice. Given these challenges, we provide the following described methods, kits, and systems which comprise:

1) Use of custom primer assays as a panel, leveraging the genomic revolution and evolutionary relationships and also testing for antibiotic resistance genes-often transferred among unrelated organisms-along with organism identification.

2) Adapted Recombinase Polymerase Assay (RPA) for this purpose, a cousin of PCR but faster, more sensitive, more robust to set up, requiring no temperature cycling, and

3) Use robotics to fully automate the setup of the RPA panel, integrating it into a fully CLIA-waived device that can be conveniently provided direct to patients, though minute-clinics or primary care physicians lacking access to a certified research laboratory capable of medical diagnostic services. Our device will interface remotely with health-care providers, giving them critical point-of-care data that they can use in making antibiotic use decisions.

RPA process employs three core enzymes—a recombinase, a single-stranded DNA-binding protein (SSB) and strand-displacing polymerase. Recombinases are capable of pairing oligonucleotide primers with homologous sequence in duplex DNA. SSB bind to displaced strands of DNA and prevent the primers from being displaced. Finally, the strand displacing polymerase begins DNA synthesis where the primer has bound to the target DNA. By using two opposing primers, much like PCR, if the target sequence is indeed present, an exponential DNA amplification reaction is initiated. There is no other sample manipulation such as thermal or chemical melting required to initiate amplification. At optimal temperatures, such as 37-42° C., the reaction progresses rapidly and results in specific DNA amplification from just a few target copies to detectable levels, typically within 10 minutes, for rapid detection of viral genomic DNA or RNA, and pathogenic bacterial genomic DNA. The described probe is a new design that uses the quencher as the 3′ blocking moiety, which is a significant improvement and simplification on the standard RPA probe which has a separate blocking moiety. The described probe is different from a standard Taq-Man probe in having an abasic site to interface with nfo nuclease during amplification cycles.

The three core RPA enzymes can be supplemented by further enzymes to provide extra functionality. Addition of exonuclease III allows the use of an exo probe for real-time, fluorescence detection akin to real-time PCR. If a reverse transcriptase that works at 37-42° C. is added then RNA can be reverse transcribed and the cDNA produced amplified all in one step. As with PCR, all forms of RPA reactions can be multiplexed by the addition of further primer/probe pairs, allowing the detection of multiple analytes or an internal control in the same tube.

### Methods

### Bacterial Strains

Streptococcus pyogenes strains, MGAS10394 (ATCC BAA-946) and MGAS6180 (ATCC BAA-1064), were obtained directly from ATCC (Manassas, Va.). Streptococcus agalactiae (NR-44140) was obtained from beiresources.org (Manassas, Va.). Streptococcus salivarius was isolated by the Kaplan lab of American University (Washington, D.C.) with patient consent for research.

### Antibiotic Testing by Broth Dilution

All bacteria were tested for their antimicrobial susceptibility by broth microdilution. Ampicillin (Cat #97061-442) was obtained from VWR (Amresco) and Erythromycin (Cat # TCE0751-5G) was obtained from VWR (TCI). Bacteria were maintained on blood agar plates at 37° C., and single colonies selected for inoculation into liquid overnight cultures in sterile Brain-Heart Infusion (BHI, VWR Cat #90003-038). For each culture, 5 mL of BHI media was inoculated in a sealed 15 ml falcon tube for overnight incubation at 37° C. (no shaking). Gentle inversion was used to mix the cultures prior to setting up the assay.

For the experiment, 5 μl of overnight culture was mixed with 5 mL of BMI media (1000× dilution) in a sterile tray and gently mixed. This dilute culture was added at 180 ml per well of a 96-well plate pre-loaded with 20 μl of antibiotic solutions ranging, for erythromycin, from 0.5 to 32 μg/ml (10×) to produce the desired final concentrations of 0.05-3.2 μg/ml. For ampicillin, the stocks were 1.25 μg/ml-80 μg/ml resulting in final concentrations of 0.125 μg/ml-8 μg/ml. The 96-well plate was then transferred to a FilterMax F5 microplate reader for a 20 hour incubation at a temperature of 37° C., with readings taken every 30 minutes. A10-second orbital shaking was performed prior to each reading.

### Specificity Testing & Adipose-Derived Stem Cell Culture

For specificity testing, human DNA was derived from primary adipose-derived cell line ASC080414A (Zen-Bio, Raleigh, N.C.) cultured in a humidified 5% CO2 incubator at 37° C. The growth media consist of Dulbecco's Modified Eagle Medium (DMEM, ThermoFisher #11965118) supplemented with 10% fetal bovine serum (ThermoFisher #10082147), 1× Penicillin/Streptomycin (ThermoFisher #15140122), and 1× Glutamax (ThermoFisher #35050061), changed every 3 days. Total DNA was purified using the Nucleospin Tissue kit (Macherey-Nagel, Duren, Germany) and quantified on a Qubit Fluorometer (ThermoFisher), which was also used to measure bacterial DNA liberated in crude lysates.

### RPA Assays

Primers and probe for the mef(A) RPA assay (Table 1) were designed following the instructions provided by TwistDx (Cambridge, UK). All primers and probes were synthesized by Integrated DNA Technologies (Coralville, Iowa). For all RPA assays the TwistDx nfo kit (TANFOO02KIT, TwistDx, Cambridge, UK) was used in agreement with manufacturer's instructions. For each reaction, a hydration mix was prepared including 4.2 L of RPA primer pair (2.1 μL/of each 10 M primer), 0.6 μL of Probe (10 M), 29.5 μL of rehydration buffer, and 13.2 μL of sample containing DNA or lysate to be tested (47.5 l total). Then the hydration mix was added to a reaction tube containing TwistAmp lyophilized enzyme pellet. The resulting mixture was mixed via pipetting 3-4 times carefully to avoid introduction of bubbles, and transferred to a qPCR 96-well plate (Agilent Cat #410088). Final concentration of primers was 420 nM and the probe was 120 nM. To activate the reaction, 2.5 μl of magnesium acetate stock solution (280 mM) was added to the caps of the 96-well plate, rapidly mixed via inversion, immediately placed in a qPCR machine (Agilent Stratagene Mx3005P). The reaction was maintained at constant temperature of 37° C. for 30 minutes, with FAM signal recorded every 30 seconds (60 total readings).

qPCR Assay

Primers F1 and R1 (Table 2) were combined at a final concentration of 176 nM with control DNA (MGAS10394) dilutions at indicated concentrations, in 1× PowerSYBR (ThermoFisher Cat #4367659) and run on an Agilent Stratagene Mx3005P. A 2-step program with 40 cycles of 30 sec at 95° C. and 1 min at 60° C. was used. The total program time was 2 hr 16 min.

PCR: 16S rDNA and mef(A)

Bacterial identification was carried out using primers 27F and 388R with 2 μl raw lysates prepared by boiling and diluting the overnight cultures. Amplification was performed in a SimpliAmp thermocycler (Applied Biosystems) with a program of 32 cycles with 95° C. for 30 sec, 52° C. for 30 sec, and 72° C. for 25 sec.

Detection of mef(A) was performed by PCR using F1 and R1 primers and 2 μl raw lysates as above. The program used was 30 cycles of 95° C. for 30 sec, 60° C. for 30 sec, and 72° C. for 10 sec.

TABLE 2
Primers and probes used in this study.

| Name | Sequence |
|---|---|
| mefA_ | 5′-GCGGTTACGCCACTTTTAGTACCAGAAGAAC |
| RPA_F1 | AGCT-3′ |
| | (SEQ ID NO: 8) |
| mefA_ | 5′-[Biotin]-TTTAGTTCCCAAACGGAGTATA |
| RPA_R1 | AGAGTGCTGCAAC-3′ |
| | (SEQ ID NO: 9) |
| mefA_ | 5′-/56-FAM/CAGGCTATAGTCAGTCTTTGCAG |
| RPA_probe | TCTATAAGC/idSp/ATATTGTTAGTCCGGC/ |
| | 3IABkFQ/-3′ |
| | (SEQ ID NO: 10) |
| 27F | 5′-AGAGTTTGATCCTGGCTCAG-3′ |
| | (SEQ ID NO: 11) |
| 388R | 5′-TGCTGCCTCCCGTAGGAGT-3′ |
| | (SEQ ID NO: 12) |

### Results

As described herein, a Taq-Man style hydrolysis probe according to one embodiment incorporating fluorophore (FAM) and quencher (Iowa Black) which doubles as a 3′ end blocker was used. Successful amplification leads to probe cleavage by Endonuclease IV (nfo) at the abasic site, separating FAM from the quencher and yielding detectable signal.

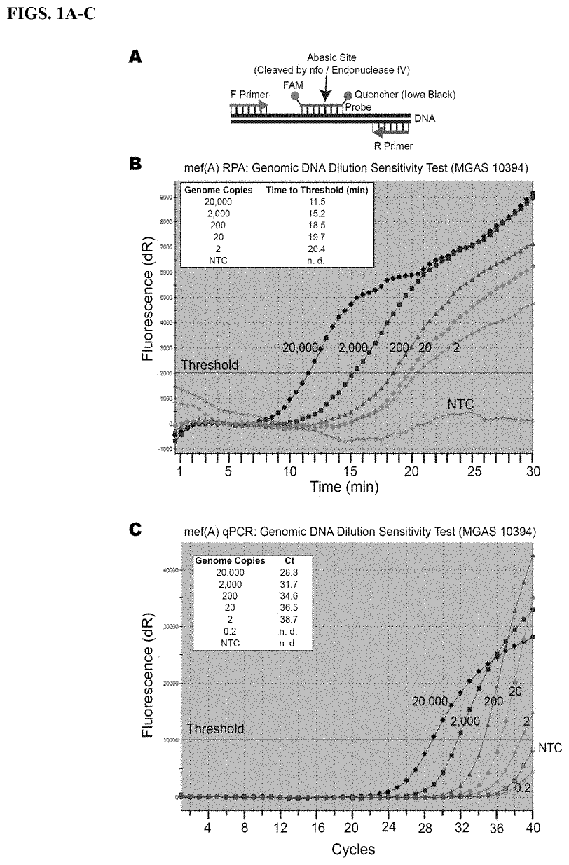

Earlier work used a quencher and FAM internally, proximal to the abasic site [9]; the present design simplifies this by using the quencher as a 3′ end blocker (FIG. 1A).

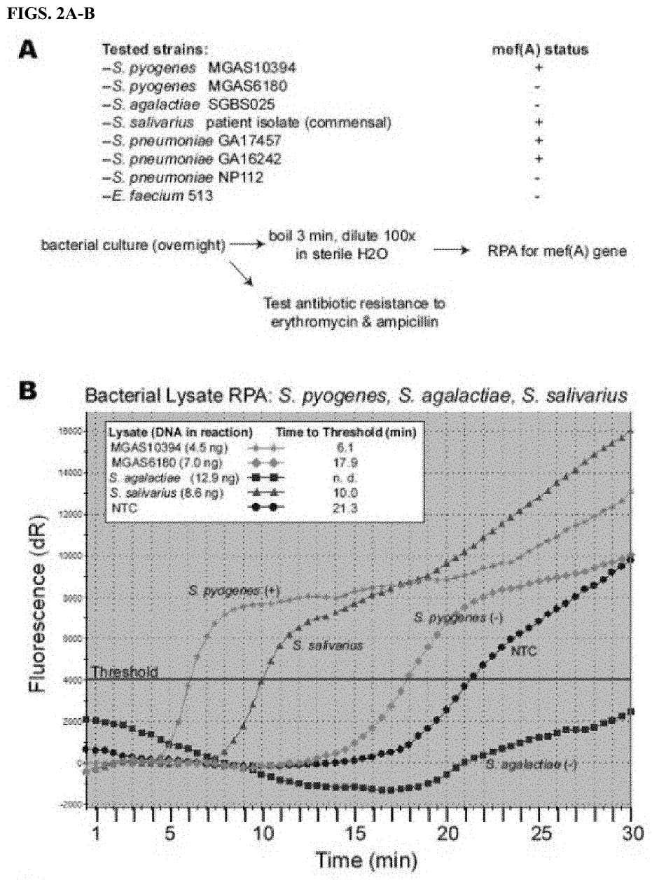

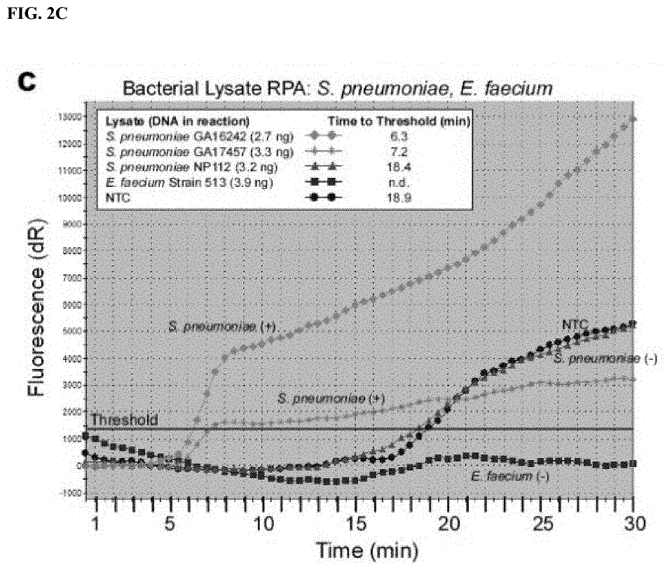

To assess assay sensitivity, serial dilution of DNA derived from mef(A)-positive Streptococcus pyogenes serotype M6 strain MGAS10394 [38] was used and found that confident detection was around 2,000 genome copies (FIG. 1B). Two-thousand genome copies corresponds to 4.3 picograms (pg) of DNA, at a concentration of 252 femtomolar (fM). While the FAM signal crosses the threshold for 200, 20, and 2 genome copies, these signals are probably nonspecific as demonstrated by negative controls showing similar late-rising (around 20 min or later) signal (FIG. 2, 3, 5). We conclude that according to this embodiment, the confident sensitivity limit of our assay is approximately 2,000 genome copies, and that detection must be recorded before 16 minutes to be considered real. The non-specific 18-20-minute signal was always easily distinguishable from real detection in our assays, which always came up quickly, around 7-10 minutes (compare FIGS. 2 and 5). The late-rising signal may be analogous to qPCR's tendency to ubiquitously amplify even no-template controls by 40 cycles. SYBR green based qPCR on the same DNA dilution series using the same primers was performed, and observed even greater sensitivity—relatively confidently down to 20 genome copies—but it was significantly slower—the run took over 2 hours (FIG. 1C). As discussed later, the 2000 genome copy threshold may help distinguish diagnostically meaningful mef(A) gene loads, rather than mere colonizers [39].

We next performed specificity testing with raw bacterial lysates from four Streptococcus strains. Mef(A) has been characterized within S. pyogenes MGAS10394 [38], a strain used as a positive control. Another Group A Strep strain, S. pyogenes MGAS6180 [40], is responsible for necrotizing fasciitis and puerperal sepsis but is negative for mef(A). An additional mef(A) negative strain was S. agalactiae, which is resistant to macrolides by a different mechanism: it hosts a target-site ribosomal methylase, ermB. Methylation of the target site in the 23S rRNA by ermB inhibits the interaction of antibiotic with the ribosome [41]. We therefore predicted—and confirmed—that this species would show an absence of mef(A) by RPA but nonetheless display robust resistance to erythromycin. Finally, we used a patient isolate of S. salivarius also as a negative control for RPA. The identities of all bacterial species were confirmed by sequencing the 16s rDNA locus (data not shown).

In one embodiment, a simple raw lysis method is employed. Individual bacterial colonies were inoculated into BHI media for overnight incubation at 37° C., followed by lysing by boiling at 95° C. for three minutes and 100-fold dilution into sterile H₂O. RPA was performed directly on this raw lysate (FIG. 2A). RPA confirmed the presence of mef(A) within MGAS10394 as expected, and its absence from MGAS6180 and S. agalactiae, but surprisingly the S. salivarius patient isolate gave a positive result (FIG. 2B).

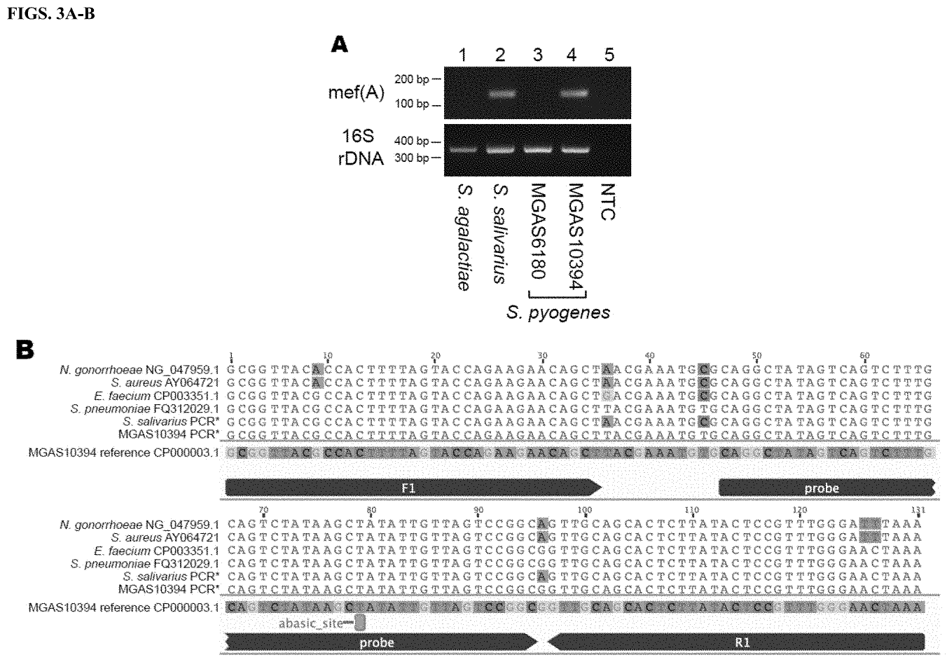

While we had not expected this commensal species to contain mef(A), we nevertheless performed PCR which confirmed the gene's presence in MGAS10394 and S. salivarius (FIG. 3A). By Sanger sequencing this product we observed that the S. salivarius gene has three single-nucleotide polymorphisms that are part of an evolutionary divergent mef(A) gene known by some authors as mef(E) and identified in Neisseria gonorrhoeae and Staphylococcus aureus [36] (FIG. 3B), suggesting that it has acquired the gene from an evolutionarily distant bacterial species and confirming that the detections constitute independent mef(A) genes, not cross contamination.

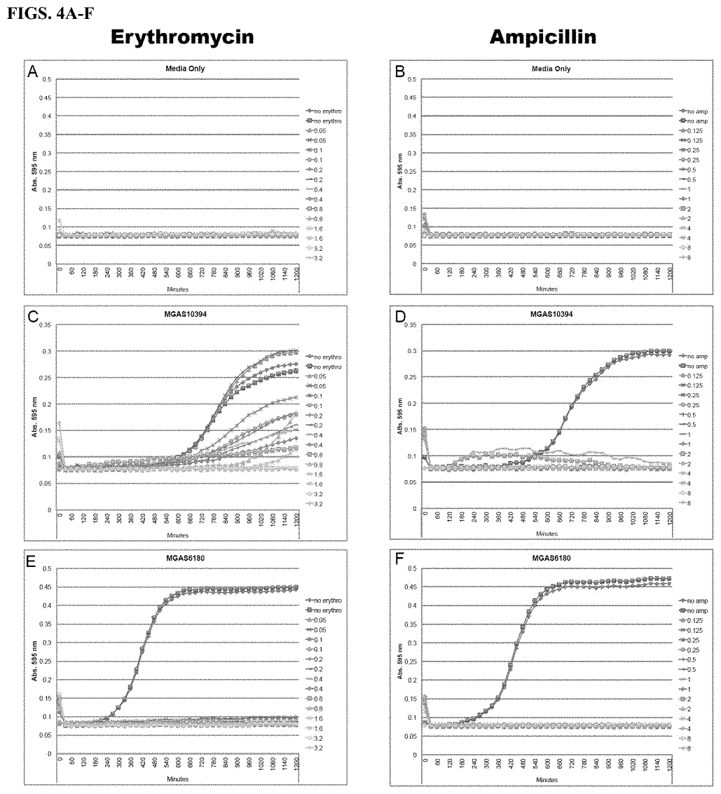

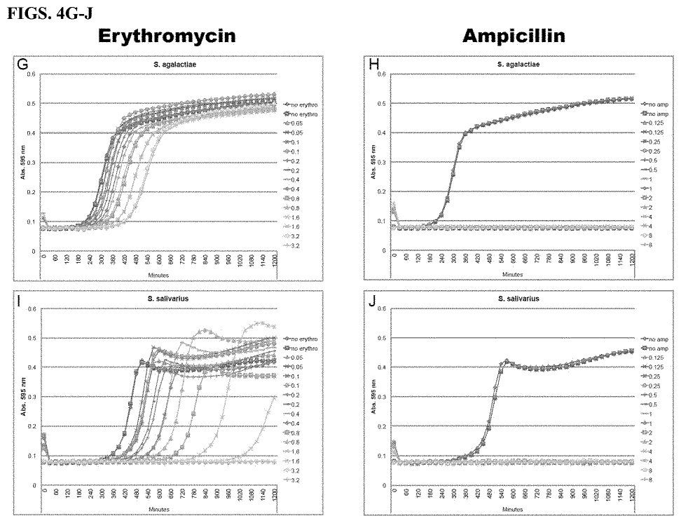

To test whether the mef(A) gene is functional, we performed broth dilution of each strain with erythromycin and ampicillin (a negative control) (FIG. 4). This confirmed that MGAS10394, S. agalactiae, and S. salivarius are all resistant to erythromycin (MIC greater than or equal to 3.2 μg/ml, Table 2) and MGAS6180 is susceptible (FIG. 4). As reported by others, ermB gives stronger erythromycin resistance than mef(A)[42, 43], with S. agalactiae giving a MIC>3.2 μg/ml (Table 3). All strains were susceptible to ampicillin as expected (FIG. 4, Table 3).

To test assay specificity, mixtures of nucleic acids was constructed as follows: A, B, and C contain 20 ng of DNA from non-mef(A) lysates (S. agalactiae plus MGAS6180) either by themselves (C) or spiked with 1.7 ng (A) or 0.34 ng (B) of MGAS10394 (mef(A)-positive). Mixes A and B represent 7.8% and 1.7% mef(A) positive, respectively. Mixes D and E tested the effect of human DNA, which might be expected to contaminate clinical samples. We therefore tested either 450 ng human DNA alone (D) or with 4.5 ng (1%) of mef(A)-positive MGAS10394 lysate (E). None of the non-specific DNA had any apparent effect on the reactions, with only E, A, and B giving specific signal and in proportion to the total mef(A) gene present in the samples (4.5 ng, 1.7 ng, and 0.34 ng, respectively) (FIG. 5). The mef(A)-negative C and D samples yielded no specific signal, giving non-specific time-to-threshold of 19.1 and 19.6 minutes, respectively (FIG. 5). Not only do these results show that the RPA assay was 100% specific and quantitative in the presence of non-specific DNA, but also functions with a wide range of total DNA in the mixture (from a few picograms, FIG. 2, to 450 ng, FIG. 5), and is robust to the conditions of raw lysate including denatured proteins, lipids, and cell wall debris.

TABLE 3
Summary of RPA, PCR, and resistance data for tested Streptococcus strains.

| | | | | MIC | MIC | |
|---|---|---|---|---|---|---|
| | | RPA from | | (μg/ml) | (μg/ml) | |
| Species | Strain | lysate (min) | PCR | (erythromycin) | (ampicillin) | Notes |
| S. pyogenes | MGAS10394 | 6.1 | + | 3.2 | <0.125 | known mef(A)+ |
| S. pyogenes | MGAS6180 | 17.9 | − | <0.05 | <0.125 | known mef(A)− |
| S. agalactiae | SGBS025 | n.d. | − | >3.2 | <0.125 | known ermB+, |
| | | | | | | mef(A)− |
| S. salivarius | Patient isolate | 10.0 | + | 3.2 | <0.125 | discovered mef(A)+ |

### Conclusions

Genomic diagnostics offer the flexibility to detect genetic material in any pathogen-bypassing the challenges associated with antibody-based assays which are much more cumbersome to produce while also being less sensitive than nucleic-acid based methods. For example, two meta-analyses of the rapid antigen-based test for group-A Streptococcal pharyngitis found an 86% sensitivity [44, 45]. This implies that 14% of true positives are mis-diagnosed by this method. Here we demonstrate a simple RPA-based genomic procedure offering flexibility and rapid detection within a similar timeframe as the rapid tests (10-15 minutes) that is very suitable to a point-of-care application. We show that we can detect down to the femtomolar (fM)/picogram (pg) range (FIG. 1B). Given the challenges of detecting lower respiratory pathogens in complex samples like saliva or sputum [46, 47], this sensitivity offers important diagnostic promise. We found that spiking in up to 100× more non-specific DNA than mef(A)+MGAS10394 DNA did not inhibit the assay, which remained extremely quantitative and specific to true target levels (FIG. 5).

Detection of antimicrobial resistance genes has been more frequently performed with Loop-mediated isothermal amplification (LAMP) rather than RPA. Examples include detection of the beta-lactamase responsible for carbapenem resistance in Acinetobacter baumannii [48, 49], the class 1 integron-integrase gene intl1 from environmental samples [50], msrA from Staphylococcus aureus [51] and mcr-1 from Enterobacteriaceae isolates [52]. In all cases, detection occurred within 20-50 minutes and generally sensitivity was in the picogram range. In contrast, the described modified RPA offers a simplified system with fewer primers that generally gives results in less than 10 minutes, which may be a critical time advantage in certain settings like clinical applications. In contrast to LAMP, genomic detection of antimicrobial resistance by RPA is still in its infancy and more progress has been made toward identifying single nucleotide polymorphisms that convey drug resistance. In one study, an HIV drug resistance allele was detected by RPA combined with an oligonucleotide ligation assay [19]. Another study identified multidrug resistant tuberculosis sequence variants using a nested RPA approach [27].

A recent study demonstrated a Thin Film Transistor sensor for RPA that significantly accelerates readout time, using pH changes during DNA amplification as an electrical signal [53]. The molecular targets in that study are beta lactamases conferring resistance to cephalosporins and carbapenems, and detection was achieved within 2-5 minutes; however those data do not include tests for specificity of the assay nor measurement of antimicrobial resistance levels in the bacteria [53]. Nevertheless these results broadly support our finding that RPA is a superior approach to genomic antimicrobial resistance testing. Innovative readout technologies hold promise to further improve temporal performance of these assays beyond the 7-10 minute detection times we demonstrate, while also providing more portable systems for point-of-care or field uses as described herein.

In this study we show that the described modified RPA is highly sensitive and specific. However, mere presence of a bacterial species does not necessarily imply infection by that organism: the diagnostic challenge is to distinguish the causative agent of disease from mere colonizer, which in many cases can be the same organism. This problem is particularly acute for lower respiratory infections, where aetiological diagnosis is only achieved 50% of the time [39]. Diagnosis of lower respiratory infection often involves analysis of sputum samples, which are notoriously non-sterile. There is debate about whether culture is even useful for these types of samples [47].

To date the most common approach to this problem is to hypothesize that true pathogens should be present at a higher level than colonizers. Therefore the standard method is to dilute the sputum sample by 10⁵ prior to culture to prevent growth of lower-abundance colonizers that would otherwise be co-cultured and might obscure the ‘true’ pathogen [54]. While this dilution may produce more clear-cut results, it also runs the risk of eliminating real pathogens or artificially attributing co-infections to a single organism. Molecular detection methods like qPCR or RPA are much more sensitive than culture methods for identifying microorganisms [39] raising the question of which is the ‘correct’ diagnostic answer. If the culture result is taken as ‘truth’ then molecular methods might be identifying many false diagnostic positives (colonizers); conversely, if qPCR is truth then culture is only 20-50% sensitive, missing (in most cases) the majority of pathogens [39]. Parsing this diagnostic tradeoff is a significant challenge for the field. Nonetheless, our RPA mef(A) assay is sensitive and specific enough to detect molecular targets at the femtomolar (fM) level, with excellent specificity (100%), enabling direct, culture-free molecular analysis of complex and difficult samples such as saliva or sputum.

Mef(A) has been found in a wide variety of bacterial hosts [36], from Neisseria gonorrhoeae [55] to Enterococcusfaecalis [56] and Streptococcus pneumoniae and pyogenes [32]. Among these genes there is significant variation at the DNA sequence level, with ˜90% identity between two main types that some authors suggest denote two genes: mef(A) and mef(E) [36], both of which nevertheless provide similar macrolide resistance. By targeting a conserved region of mef(A) (FIG. 3), our assay is capable of detecting both the mef(A)-type and mef(E)-type genes, both of which we functionally validated (FIG. 4) highlighting the wide diagnostic potential of the method. We anticipate this assay will become an important tool in the diagnostic toolbox, offering physicians and scientists alike a rapid, accurate measure of antimicrobial resistance, whether hosted in the upper (S. pyogenes [32] or S. salivarius [37]) or lower respiratory tract (Streptococcus pneumoniae [32] or Staphylococcus aureus [57] or others), or in other regions of the human microbiome.

Resistance to antibiotics is a widely recognized health emergency. The spread of antibiotic-resistant Streptococcus pneumoniae is considered epidemic, erythromycin-resistant Group A Streptococcus and Clindamycin-resistant Group B Streptococcus are listed as concerning threats of antibiotic resistance, and Cabapenems-resistant Enterobacteriaceae (CRE) is resistant to virtually all antibiotics jeopardizing public health and safety. Antimicrobial resistance has been identified in all bacterial, fungal, viral and parasitic treatments known to date. The CDC and WHO have emphasized the possibility of a future post-antibiotic world that eradicates the medical advances made in the last 100 years. Thus, antibiotic resistance is now considered the most significant threat to human health worldwide since non-threatening infections that were once treated with antibiotics may no longer be controllable. To remedy the problem of respiratory misdiagnosis and overuse of antibiotics, the Institute of Medicine (IOM) recommends that patients become full partners in their own care, and that is the major goal of the diagnostic technology described herein.

The described methods and device addresses antimicrobial resistance in two ways: 1) accurate diagnostics enabling patients to avoid antibiotics in the case of viral infections, and 2) detection of specific pathogen-hosted antibiotic resistance genes, allowing the correct-effective-antibiotics to be utilized. The latter is important because resistance genes can be passed between bacteria independently of their genetic identity—through horizontal gene transfer (HGT) in the form of transposons, plasmids, or bacteriophages.

As described, a novel RPA assay for antimicrobial resistance gene mef(A), an element giving resistance to erythromycin and common in Streptococcus A strains, is provided. The assay was able to detect of mef(A) in raw lysates of Streptococcus pyogenes, S. pneumoniae, S. salivarius, and Enterococcus faecium bacterial lysates within 7-10 minutes, and confirmed gene presence with traditional PCR and sequencing. Furthermore, the RPA detection of mef(A) accurately predicted real antimicrobial resistance assessed by traditional culture methods, and that the assay is robust to high levels of spiked-in non-specific nucleic acid contaminant. The assay was also unaffected by single-nucleotide polymorphisms within divergent mef(A) genes, strengthening its utility as a robust diagnostic tool. This finding opens the door to implementation of rapid genomic diagnostics in a clinical setting, while providing researchers a rapid, cost-effective tool to track antibiotic resistance in both pathogens and commensal strains.

To assess assay sensitivity, a serial dilution of DNA derived from mef(A)-positive Streptococcus pyogenes serotype M6 strain MGAS10394 was used, and found that confident detection was around 2,000 genome copies (FIG. 1B). Two-thousand genome copies corresponds to 4.3 picograms (pg) of DNA, at a concentration of 252 femtomolar (fM). While the FAM signal crosses the threshold for 200, 20, and 2 genome copies, these signals are probably nonspecific as demonstrated by negative controls showing similar late-rising (around 20 min or later) signal (SEE FIGS. 2 and 5). We conclude that the confident sensitivity limit of our assay is approximately 2,000 genome copies, and that detection must be recorded before 16 minutes to be considered real. The non-specific 18-20-minute signal was always easily distinguishable from real detection in our assays, which always came up quickly, around 7-10 minutes (compare FIGS. 2B,2C, and FIG. 5). We suggest the late-rising signal is analogous to qPCR's tendency to ubiquitously amplify even no-template controls by 40 cycles. We performed SYBR green based qPCR on the same DNA dilution series using the same primers, and observed even greater sensitivity-relatively confidently down to 20 genome copies—but it was significantly slower—the run took over 2 hours (FIG. 1C). The 2000 genome copy threshold may help distinguish diagnostically meaningful mef(A) gene loads, rather than mere colonizers.

We also performed specificity testing with raw bacterial lysates from eight bacterial strains. Mef(A) is present within genomes of Group A Strep strain S. pyogenes MGAS10394, as well as S. pneumoniae strains GA17457 (GenBank accession AILS00000000.1) and GA16242 (GenBank accession AGPE00000000.1). Known mef(A) negative strains include S. pyogenes MGAS6180 responsible for necrotizing fasciitis and puerperal sepsis, Enterococcus faecium Strain 513 (GenBank accession AMBG00000000.1), S. pneumoniae strain NP112 (GenBank accession AGQF00000000.1) and S. agalactiae SGBS025 (GenBank accession AUWE00000000.1). Streptococcus agalactiae is resistant to macrolides by a different mechanism than mef(A): it hosts a target-site ribosomal methylase, ermB. Methylation of the target site in the 23S rRNA by ermB inhibits the interaction of antibiotic with the ribosome. We show that this species would exhibit an absence of mef(A) by RPA but nonetheless display robust resistance to erythromycin. Finally, we tested a patient isolate of S. salivarius. The identities of S. salivarius, S. agalactiae, and S. pyogenes strains were confirmed by sequencing the 16s rDNA locus (data not provided).

To evaluate assay specificity, we constructed mixtures of nucleic acids as follows: A, B, and C contain 20 ng of DNA from non-mef(A) lysates (S. agalactiae plus MGAS6180) either by themselves (C) or spiked with 1.7 ng (A) or 0.34 ng (B) of MGAS10394 (mef(A)-positive). Mixes A and B represent 7.8% and 1.7% mef(A) positive, respectively. Mixes D and E tested the effect of human DNA, which might be expected to contaminate clinical samples. We therefore tested either 450 ng human DNA alone (D) or with 4.5 ng (1%) of mef(A)-positive MGAS10394 lysate (E). None of the non-specific DNA had any apparent effect on the reactions, with only E, A, and B giving specific signal and in proportion to the total mef(A) gene present in the samples (4.5 ng, 1.7 ng, and 0.34 ng, respectively) (FIG. 5). The mef(A)-negative C and D samples yielded no specific signal, giving non-specific time-to-threshold of 19.1 and 19.6 minutes, respectively (FIG. 5). Not only do these results show that the RPA assay was 100% specific and quantitative in the presence of non-specific DNA, but also functions with a wide range of total DNA in the mixture, and is robust to the conditions of raw lysate including denatured proteins, lipids, and cell wall debris.

RPA is considerably more robust to traditional PCR inhibitors, making it possible to eliminate complex nucleic acid purifications. In fact, RPA has been demonstrated to detect Chlamydia directly from human urine, which is notorious for containing PCR inhibitors. We show that detection of mef(A) accurately predicts real antimicrobial resistance assessed by traditional culture methods, and that the assay is robust to high levels of spiked-in non-specific nucleic acid contaminant. The assay was unaffected by single-nucleotide polymorphisms within divergent mef(A) genes, strengthening its utility as a robust diagnostic tool. This is important because many bacteria and viruses can exist commensally without causing disease (colonizers). The diagnostic challenge created by this includes the risk of false-positive (Type I) error, which is always balanced by the need to avoid false-negative (Type II) error in which true pathogens go undetected. The value of RPA has not yet been realized in the clinical setting.

### Respiratory Infections

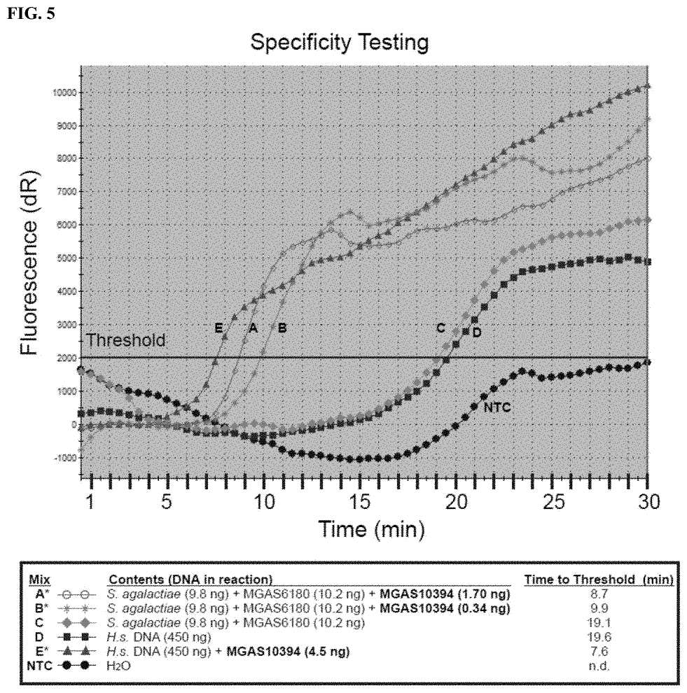

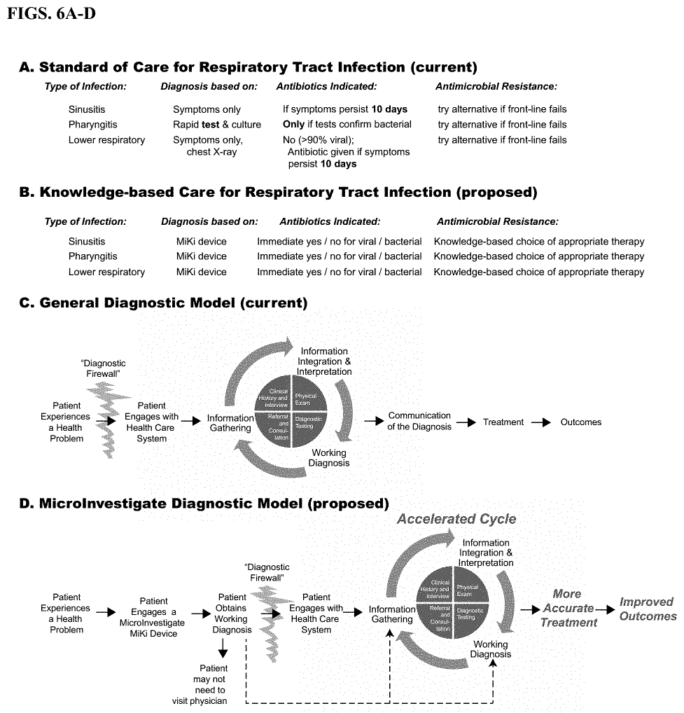

In another aspect, we provide knowledge-based diagnostic information (FIG. 6A, B) that ultimately will shape healthcare (FIG. 6C, D). Based upon published data on molecular diagnostic sensitivity and similar results from the described RPA assay (FIG. 1A), all three respiratory infection types (sinusitis, pharyngitis, and bronchitis) can be addressed using the described method and device. Using the quantitative aspects of the molecular assays to provide likelihood estimates of pathogenicity, not just colonization, which helps to overcome common problems with culture-based analysis of sputum, for example, being heavily contaminated with normal bacterial flora.

As described, one aspect is to focus initially on diagnosing respiratory diseases because of their unique combination of broad health implications, high morbidity, and diagnostic challenge. Indeed, lower respiratory infections constitute the leading cause of death by infectious diseases worldwide. For these types of infections, no etiological agent is even identified 50% of the time. Respiratory diseases are particularly challenging to diagnose correctly because only a few rapid tests are available, culture is slow, and often samples such as sputum are uninformative due to culture contamination. Broadly speaking, respiratory infection can be categorized as pharyngitis/upper (infection of the pharynx at the back of the throat), sinusitis (infection of nasal sinuses), or bronchitis/lower respiratory infection (infection of the bronchial tubes in the lung). Within an infection area (upper, lower, or sinus), symptoms of bacterial or viral infection often overlap, providing only poor diagnostic power. Upper respiratory infection (pharyngitis) tends to be self-limiting and relatively benign, yet still have a significant morbidity, with reports showing tens of thousands of lost school and workdays among college students alone. Meanwhile, rampant unnecessary prescription of antibiotics for upper respiratory tract infection creates negative health consequences both for individuals and the environment. It has been estimated that 7.4 million antibiotic prescriptions were written by physicians for respiratory infections in 2001, but Bertino (2002) estimated that only 5% of those infections are bacterial, suggesting that antibiotic therapy is incorrectly prescribed 95% of the time. These data implicate upper respiratory infections as important contributors to antibiotic overuse, the evolution of antibiotic resistance, and a key area of need for diagnostic improvements.

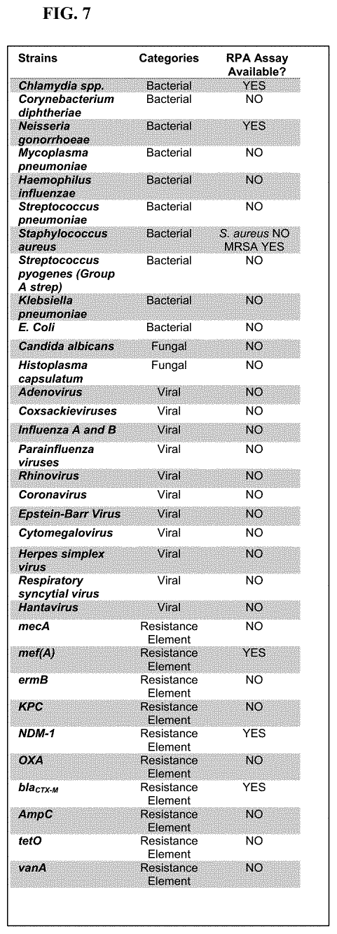

Salivary diagnostics, or the use of saliva in diagnostics, is an exciting emerging field of research. To date, most infectious diseases have been detected in saliva by the presence of antibodies rather than nucleic acids. Common respiratory pathogens need to be diagnosed quickly, prior to a strong antibody response, so nucleic acid detection offers distinct advantages for point-of-care respiratory diagnosis. Multiple human pathogens are detectable in saliva by nucleic acid testing: Human Immunodeficiency Virus (HIV), Human Papilloma Virus (HPV), Nisseria gonorrhoeae, Mycoplasma pneumoniae, Candida albicans and even Zika. Several of these pathogens are suitable for assay development (FIG. 7). However to date, no systematic analysis of nucleic acid-based diagnosis of respiratory pathogens from saliva compared to nasal or throat swabs has been performed. Many respiratory pathogens will be detectable owing to the sensitivity of RPA and the fact that the oral cavity is in relatively close communion with both upper and lower airways. Therefore, we will examine clinical samples in which patient-matched throat swabs, nasopharyngeal swabs, and saliva are compared directly using extremely sensitive nucleic acid-based amplification methods. Because the described mef(A) assay can detect down to femotomolar (picogram) levels of target DNA (FIG. 2), we anticipate significant improvements in the ability to detect even low-level, hard-to-detect deep lung infections in salivary samples. We have also developed methods to distinguish colonizers, normal members of the microbiota, from true pathogens, essential for detection methods with this high sensitivity, as described below.

In one embodiment, the respiratory panel will consist of the selected 33 targets in FIG. 7. These 24 species and 10 resistance elements cover the majority of human respiratory infections and many of the resistance elements of severe health concern. Assay design primarily consists of leveraging genomic sequence data. This method has already been tested and confirmed via our first assay for mef(A), the Streptococcus pyogenes-associated antibiotic resistance gene. This novel assay both tracks real antimicrobial resistance tested in the laboratory while giving results in under 10 minutes, but it also uncovered an example of antimicrobial resistance in a non-pathogen, Streptococcus salivarius. While antimicrobial resistance genes have been previously shown to exist within S. salivarius, we are the first to find it using RPA. This finding highlights the power of RPA to provide both clinically important resistance information and insight into new cases of antimicrobial resistance lurking in unexpected places.

For assay design more generally, for each target (FIG. 7), genomic sequences are downloaded from Genbank and species specificity confirmed with Basic Local Alignment Search Tool (BLAST) against the Genbank database (the non-redundant, or nr database). Alignments of representative sequences from phylogenetically diverse taxa are generated locally using MAFFT implemented within the Geneious software suite (www.biomatters.com) and conserved regions identified by manual inspection. Multiple primers are designed and ordered for testing (from Integrated DNA Technologies, Inc.); in the case of mef(A), we designed 4 pairs (8 total). Primer validation was performed using purified bacterial DNA ordered from ATCC. In one embodiment, the assay panel comprises one or more of the following primer pairs:

1)
(SEQ ID NO: 13)

| | |
|---|---|
| ErmB_F1 | ATTCACCAAGATATTCTACAGTTTCAATTC |

(SEQ ID NO: 14)

| | |
|---|---|
| ErmB_F2 | TTGAATTAGACAGTCATCTATTCAACTTATC |

(SEQ ID NO: 15)

| | |
|---|---|
| ErmB_R1 | CACTGTTTACTTTTGGTTTAGGATGAAAGC |

(SEQ ID NO: 16)

| | |
|---|---|
| ErmB_R2 | CCAATATTTATCTGGAACATCTGTGGTATG |

2)
(SEQ ID NO: 17)

| | |
|---|---|
| mecA_F1 | ATGGAGTTGAAAGATTTCTTGATTCCTCAAG |

(SEQ ID NO: 18)

| | |
|---|---|
| mecA_F2 | CAATTAGTATGGACGATTTAGAAGAAAGAGG |

(SEQ ID NO: 19)

| | |
|---|---|
| mecA-R1 | ACATATCACCAAACTCTGCTAAATCTTCAAG |

(SEQ ID NO: 20)

| | |
|---|---|
| mecA_R2 | TCCAATTTTTCATGAGCTTTGACATCTCCC |

3)
(SEQ ID NO: 21)

| | |
|---|---|
| AmpC_F1 | TGAGCTAGGATCGGTTAGTAAGACGTTTAAC |

(SEQ ID NO: 22)

| | |
|---|---|
| AmpC_F2 | TATTATTTCACCTGGGGTAAAGCCGATATCG |

(SEQ ID NO: 23)

| | |
|---|---|
| AmpC_R1 | ATGCAGTAATGCGGCTTTATCCCTAACGTC |

(SEQ ID NO: 24)

| | |
|---|---|
| AmpC_R2 | GTCTGGTCATTGCCTCTTCGTAACTCATTC |

4)
(SEQ ID NO: 25)

| | |
|---|---|
| blaCTX-M_F1 | GACGTACAGCAAAAACTTGCCGAATTAGAG |

(SEQ ID NO: 26)

| | |
|---|---|
| blaCTX-M_F2 | AGGCAGACTGGGTGTGGCATTGATTAACAC |

(SEQ ID NO: 27)

| | |
|---|---|
| blaCTX-M_R1 | TCGCTGATTTAACAGATTCGGTTCGCTTTC |

(SEQ ID NO: 28)

| | |
|---|---|
| blaCTX-M_R2 | CCGCAATCGGATTATAGTTAACAAGGTCAG |

5)
(SEQ ID NO: 29)

| | |
|---|---|
| KPC_F1 | GATACCGGCTCAGGCGCAACTGTAAGTTAC |

(SEQ ID NO: 30)

| | |
|---|---|
| KPC_F2 | TCGCTAAACTCGAACAGGACTTTGGCGGCT |

(SEQ ID NO: 31)

| | |
|---|---|
| KPC_R1 | CATCCGTTACGGCAAAAATGCGCTGGTTCC |

(SEQ ID NO: 32)

| | |
|---|---|
| KPC_R2 | CAAATTGGCGGCGGCGTTATCACTGTATTG |

6)
(SEQ ID NO: 33)

| | |
|---|---|
| NDM-1_F1 | GATCCCAACGGTGATATTGTCACTGGTGTG |

(SEQ ID NO: 34)

| | |
|---|---|
| NDM-1_F2 | TAGTGCTCAGTGTCGGCATCACCGAGATTG |

(SEQ ID NO: 35)

| | |
|---|---|
| NDM-1_R1 | CGACTTATGCCAATGCGTTGTCGAACCAGC |

(SEQ ID NO: 36)

| | |
|---|---|
| NDM-1_R2 | CAGATCCTCAACTGGATCAAGCAGGAGATC |

7)
(SEQ ID NO: 37)

| | |
|---|---|
| OXA_F1 | GAATGGAGATCTGGAACAGCAATCATACAC |

(SEQ ID NO: 38)

| | |
|---|---|
| OXA_F2 | TCGCATTATCACTTATGGCATTTGATGCGG |

(SEQ ID NO: 39)

| | |
|---|---|
| OXA_R1 | CCATGCTTCTGTTAATCCGTTGTTTCTTTC |

(SEQ ID NO: 40)

| | |
|---|---|
| OXA_R2 | CCAGAGAAGTCTTGATTTCCATAATCAAAATC |

8)
(SEQ ID NO: 41)

| | |
|---|---|
| tetO_F1 | GACAGATACAATGAATTTGGAGCGTCAAAG |

(SEQ ID NO: 42)

| | |
|---|---|
| tetO_F2 | GTTTATTGTATACCAGTGGTGCAATTGCAG |

(SEQ ID NO: 43)

| | |
|---|---|
| tetO_R1 | CATTATCTGTAGTGCATGAAACAGTATACGG |

(SEQ ID NO: 44)

| | |
|---|---|
| tetO_R2 | CCATCCTTTGCAGAAACTAATAATACTGCTC |

9)
(SEQ ID NO: 45)

| | |
|---|---|
| vanA_F1 | CGAATTGGACTACGCAATTGAATCGGCAAG |

(SEQ ID NO: 46)

| | |
|---|---|
| vanA_F2 | TTAATTGAGCAGGCTGTTTCGGGCTGTGAG |

(SEQ ID NO: 47)

| | |
|---|---|
| vanA_R1 | GTAAAAACATATCCACACGGGCTAGACCTC |

(SEQ ID NO: 48)

| | |
|---|---|
| vanA_R2 | CTGCGGGAACGGTTATAACTGCGTTTTCAG |

10)
(SEQ ID NO: 49)

| | |
|---|---|
| TetM_F1 | CGCTTCTACGATATTACGTGGATTCTACGA |

(SEQ ID NO: 50)

| | |
|---|---|
| TetM_F2 | GTGCACTGTTGCAAGAAAAGTATCATGTGG |

(SEQ ID NO: 51)

| | |
|---|---|
| TetM_R1 | GATTGATTTAAGTATCCAAGAGAAACCGAGC |

(SEQ ID NO: 52)

| | |
|---|---|
| TetM_R2 | CAGGGCTATAGTATAAGCCATACTTAAAACAG |

11)
(SEQ ID NO: 53)

| | |
|---|---|
| ftsl_F1 | GGTCAAATACTACAGATGCAACTTAAACGG |

(SEQ ID NO: 54)

| | |
|---|---|
| ftsl_F2 | GCTTTTTACTTTTCCATTGCTGTAACCACTC |

(SEQ ID NO: 55)

| | |
|---|---|
| ftsl_R1 | TCGGTCTTCTTTTCCTCTATCTGCGCATTG |

(SEQ ID NO: 56)

| | |
|---|---|
| ftsl_R2 | GCAATTTCTTTCAAACGTTCTGCACGTAATAG |

12)
(SEQ ID NO: 57)

| | |
|---|---|
| mprF_F1 | CATTGCTAATTGTATTCCATGTTTTCGATGC |

(SEQ ID NO: 58)

| | |
|---|---|
| mprF_F2 | GGCGTTAGAGCAATGGTTTATAAAAACTATAC |

(SEQ ID NO: 59)

| | |
|---|---|
| mprF_R1 | GAATAAAGCTGACTAAACCTGATAATGCAG |

(SEQ ID NO: 60)

| | |
|---|---|
| mprF_R2 | CCGGTACAAAATAGTACGCAAAACGATATA |

13)
(SEQ ID NO: 61)

| | |
|---|---|
| mcr-1_F1 | GATAAAATCAGCCAAACCTATCCCATCGCG |

(SEQ ID NO: 62)

| | |
|---|---|
| mcr-1_F2 | CGCTATGTGCTAAAGCCTGTGTTGATTTTG |

(SEQ ID NO: 63)

| | |
|---|---|
| mcr-1_R1 | CGCATGATAAACGCTGCGTTTAATAGATCC |

(SEQ ID NO: 64)

| | |
|---|---|
| mcr-1_R2 | CCTTAACAAAAGCCACAAGCAAACTTGGTA |

14)
(SEQ ID NO: 65)

| | |
|---|---|
| QnrA_F1 | GCTTTTATCAGTGTGACTTCAGCCACTGTC |

(SEQ ID NO: 66)

| | |
|---|---|
| QnrA_F2 | CAGCAAGAGGATTTCTCACGCCAGGATTTG |

(SEQ ID NO: 67)

| | |
|---|---|
| QnrA_R1 | CATTGCTCCAGTTGTTTTCAAACAGCTCGC |

(SEQ ID NO: 68)

| | |
|---|---|
| QnrA_R2 | CAGAAGTACATCTTATGGCTGACTTGATTG |

15)
(SEQ ID NO: 69)

| | |
|---|---|
| gyrA_F1 | GTGATCACCGAGTTGCCGTATCAGGTCAAC |

(SEQ ID NO: 70)

| | |
|---|---|
| gyrA_F2 | CAAGCTGGCCGGCATTTCCAACATTGAGGA |

(SEQ ID NO: 71)

| | |
|---|---|
| gyrA_R1 | GGTCAACGTAATAGCGGATCAGCTGGTCCA |

(SEQ ID NO: 72)

| | |
|---|---|
| gyrA_R2 | GTCTGCAGCTGGGTGTGCTTGTAAAGGTTAT |

16)
(SEQ ID NO: 73)

| | |
|---|---|
| gyrB_F1 | GTGTGAAGGGCTTCGTCGAGTACATCAACA |

(SEQ ID NO: 74)

| | |
|---|---|
| gyrB_F2 | CACGGTCGAATATCACTACGACATCCTCGC |

(SEQ ID NO: 75)

| | |
|---|---|
| gyrB_R1 | GATGTACTTGTTGATGACGCGCGTCATCGC |

(SEQ ID NO: 76)

| | |
|---|---|
| gyrB_R2 | CAGAAGAAACCAGCTTGTCCTTCGTCTGCG |

17)
(SEQ ID NO: 77)

| | |
|---|---|
| parE_F1 | GTTGAAATCACTCGCGATGGTGCAATCTAC |

(SEQ ID NO: 78)

| | |
|---|---|
| parE_F2 | GTACAGCACCCAAGTCTAAAACAGGTACCA |

(SEQ ID NO: 79)

| | |
|---|---|
| parE_R1 | CTTCCACTTGGAAACCATTATCTTCTCCTT |

(SEQ ID NO: 80)

| | |
|---|---|
| parE_R2 | GGTCTCCTTGTCTTCGTTAAGATAAGAAAC |

18)
(SEQ ID NO: 81)

| | |
|---|---|
| parC_F1 | CTATCTATGATGCCATGGTTCGTATGTCAC |

(SEQ ID NO: 82)

| | |
|---|---|
| parC_F2 | GGTAACATCATGGGGAATTTCCACCCACAC |

(SEQ ID NO: 83)

| | |
|---|---|
| parC_R1 | GCAATCTCAGACAAACGCGCCTCAGTATAA |

(SEQ ID NO: 84)

| | |
|---|---|
| parC_R2 | CAGTCGAACCATTGACCAAGAGGTTTGGAA |

19)
(SEQ ID NO: 85)

| | |
|---|---|
| rpoB_F1 | GTACTTCGACGAGACCATTGACAAGTCCAC |

(SEQ ID NO: 86)

| | |
|---|---|
| rpoB_F2 | GATGATGACCGAGAAGGGCACGTTCATCAT |

(SEQ ID NO: 87)

| | |
|---|---|
| rpoB_R1 | GAACAAGTTTTCCAACAGCGTCTGCGCTGA |

(SEQ ID NO: 88)

| | |
|---|---|
| rpoB_R2 | CAGCCCGAGCTTCTTGTTGACCTTATAGCG |

20)
(SEQ ID NO: 89)

| | |
|---|---|
| cfr_F1 | GAAGTATCAAAGAATGAGAGAGTAGAAACG |

(SEQ ID NO: 90)

| | |
|---|---|
| cfr_F2 | GGATATGAAGGTTCTTCCAAAATTACTTAG |

(SEQ ID NO: 91)

| | |
|---|---|
| cfr_R1 | AGAGCTTCACCCATTCCCATAAAAGAAATG |

(SEQ ID NO: 92)

| | |
|---|---|
| cfr_R2 | GGAAGTATAAAACTTGATCTGTTATCTCATC |

21)
(SEQ ID NO: 93)

| | |
|---|---|
| MefA_F1 | GCGGTTACGCCACTTTTAGTACCAGAAGAACAGCT |

(SEQ ID NO: 94)

| | |
|---|---|
| MefA_R1 | TTTAGTTCCCAAACGGAGTATAAGAGTGCTGCAAC. |

In one embodiment, the assay panel comprises one or more of the following probes:

1) ErmB_quench_probe:
(SEQ ID NO: 95)
/5HEX/TACCTTGGATATTCACCGAACACTAGGGTTG/idSp/
TCTTGCACACTCAAG/3IABkFQ/
100 nm HPLC
2) mecA_quench_probe:
(SEQ ID NO: 96)
/5Cy3/TTTAAAGATAGTGGTATGCTTAGTTTTCGA/idSp/
TGACTCCACGCAAGG/3IABkRQ/
100 nm HPLC
3) AmpC_quench_probe:
(SEQ ID NO: 97)
/5Cy5/TGGCCAGAACTGACAGGCAAACAGTGGCAG/idSp/
GTATCCGCCTGCTGC/3IABkRQ/
100 nm HPLC
4) blaCTX-M_quench_probe:
(SEQ ID NO: 98)
/56-
FAM/TTCGCAAATACTTTATCGTGCTGATGAGCG/idSp/
TTTGCGATGTGCAGC/3IABkFQ/
100 nm HPLC
5) KPC_quench_probe
(SEQ ID NO: 99)
/5HEX/TGCTGCCGCTGTGCTGGCTCGCAGCCAGCA/idSp/
CAGGCCGGCTTGCTG/3IABkFQ/
100 nm HPLC
6) NDM-1_quench_probe
(SEQ ID NO: 100)
/5Cy3/TTGCTGGTTCGACCCAGCCATTGGCGGCGA/idSp/
AGTCAGGCTGTGTTG/3IABkRQ/
100 nm HPLC
7) OXA_quench_probe
(SEQ ID NO: 101)
/5Cy5/TTGGGTTTCGCAAGAAATAACCCAAAAAAT/idSp/
GGATTAAATAAAATC/3IABkRQ/
100 nm HPLC
8) tetO_quench_probe
(SEQ ID NO: 102)
/56-
FAM/TGGGAGGATGTAAAAGTCAACATTATAGAT/idSp/
CGCCAGGCCATATGG/3IABkFQ/
100 nm HPLC
9) vanA_quench_probe
(SEQ ID NO: 103)
/5HEX/TCAGGCTGCAGTACGGAATCTTTCGTATTC/idSp/
TCAGGAAGTCGAGCC/3IABkFQ/
100 nm HPLC
10) TetM_quench_probe
(SEQ ID NO: 104)
/5HEX/TGCCGCCAAATCCTTTCTGGGCTTCCATTG/idSp/
TTTATCTGTATCACC/3IABkFQ/
100 nm HPLC
11) ftsl_quench_probe
(SEQ ID NO: 105)
/5Cy3/TATTATTTTTATGCAGACCAAGCTCTTGCA/idSp/
GTGCAGAATGATTTG/3IABkRQ/
100 nm HPLC
12) mprF_quench_probe
(SEQ ID NO: 106)
/5Cy5/TGTGTTGAATGGTTAGCAGCTGCAGTTGTA/idSp/
TATATTTCTGTGGTG/3IABkRQ/
100 nm HPLC
13) Mcr-1_quench_probe
(SEQ ID NO: 107)
/56-FAM/TATTTTACTGACACTTATGGCACGGTCTAT/idSp/
ATACGACCATGCTCC/3IABkFQ/
100 nm HPLC
14) Qnr_quench_probe
(SEQ ID NO: 108)
/5HEX/TTCAGCTATGCCGATCTGCGCGATGCCAGT/idSp/
TCAAGGCCTGCCGTC/3IABkFQ/
100 nm HPLC
15) gyrA_quench_probe
(SEQ ID NO: 109)
/5Cy3/TCTAGCGATCGGGTCGGTTTACGCATCGTC/idSp/
TCGAGATCAAGCGCG/3IABkRQ/
100 nm HPLC
16) gyrB_quench_probe
(SEQ ID NO: 110)
/5Cy5/TGCAGTGGAACGACAGCTACAACGAGAACG/idSp/
GCTGTGCTTCACCAAC/3IABkRQ/
100n m HPLC
17) parE_quench_probe
(SEQ ID NO: 111)
/56-
FAM/TCTTGAAAAATGTGACCTTGTCCTTGACGG/idSp/
CAAGCGAACAGATGA/3IABkFQ/
100 nm HPLC
18) parC_quench_probe
(SEQ ID NO: 112)
/5HEX/TGAAATGCACGGTAATAACGGTTCTATGGA/idSp/
GGAGATCCTCCTGCG/3IABkFQ/
100 nm HPLC
19) rpoB_quench_probe
(SEQ ID NO: 113)
/5Cy3/TGCGCATCGACCGCAAACGCCGGCAACCGG/idSp/
CACCGTGCTGCTCAAG/3IABkRQ/
100 nm HPLC
20) cfr_quench_probe
(SEQ ID NO: 114)
/5Cy5/TCATCACAATGCGGATGTAATTTTGGGTGT/idSp/
AATTTTGTGCTACAG/3IABkRQ/
100 nm HPLC
21) MefA_quench_probe
(SEQ ID NO: 115)
/56-FAM/CAGGCTATAGTCAGTCTTTGCAGTCTATAAGC/idSp/
ATATTGTTAGTCCGGC/3IABkFQ/

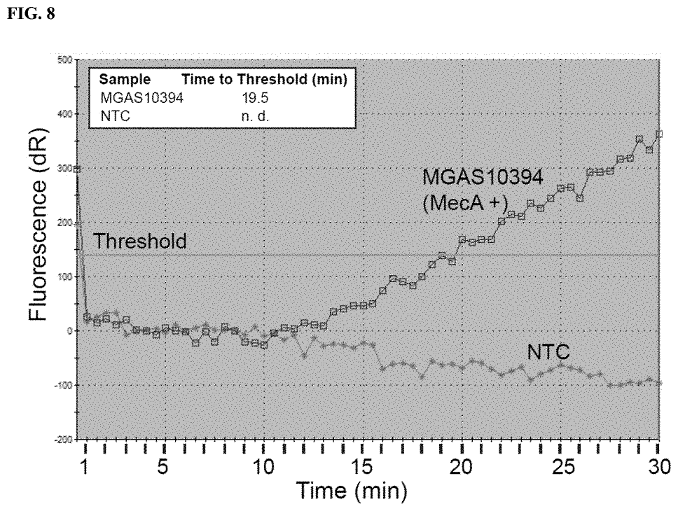

In one embodiment, the mecA resistance element is detected, as shown in FIG. 8. MecA RPA assay using raw bacterial lysate (purple line marked with squares) or no-template control (NTC, green line marked with asterisks). Overnight MGAS10394 culture was boiled for 3 min at 95 degrees and diluted 100× in purified water, then used in assay. Time to threshold was 19.5 min.

In one embodiment, more than of the target nucleic acids is amplified using a multiplex assay. Multiplex assays, such as multiplex qPCR, are an efficient and cost effective solution for overcoming the challenges of limited samples and costly analysis. Successful multiplex assay enables the amplification of more than one target in a single reaction using different reporters with distinct fluorescent spectra.

We also demonstrated RPA can be used on raw bacterial lysates after brief (3 min) boiling lysis of live bacteria, an important simplifying innovation for implementation within a robotic kiosk, and consistent with, for example, direct detection of genomic targets from unpurified urine by RPA. Also, many viruses are RNA-based and detection of RNA involves reverse transcription into complementary DNA (cDNA). By incorporating reverse transcriptase (RT) into the RPA methods and device, a single unified methodology for RPA of any pathogen, including RNA viruses, is obtained.

The standard approach for normalization to avoid Type I error is to assume etiological agents are present at higher levels than colonizers in the sick person, so quantitation is essential, not just high sensitivity, which might be actually misleading. Quantitative information will also enable a patient to obtain baseline ‘healthy’ status and also to monitor resolution of infections in real-time, for example on an antimicrobial therapy. To accomplish this, we will quantitate the overall bacterial load and normalize the level of each detected species to this value. Akin to the ΔΔCt method for qPCR, an RPA assay targeting a universal bacterial 16S rRNA sequence will measure total bacterial loads (well characterized already for qPCR), and the difference in time-to-detection of the putative pathogen (in A-minutes, Δₘᵢₙ) will be used to infer levels and concern levels for various organisms detected. The Δₘᵢₙ threshold for each organism will be established empirically as we build out and test the assay panel with clinical samples, and will vary by pathogen and sample type (saliva, throat swab, or nasal swab). Ultimately the Δₘᵢₙ threshold will be used to distinguish colonization from infection.

While bacterial normalization is straightforward, viral load normalization is more complex to measure. The literature reports up to 68% of healthy individuals harbor viral ‘commensals’ but the fact that the species are widely diverse, not present in 32% of individuals, and lack a conserved genomic sequence makes viral normalization an open research question. However, we will normalize to bacterial 16S rRNA loads, a well-validated measure, as a proxy for total microbial content.

There has been a recent focus on the reservoirs of antimicrobial resistance genes (‘resistomes’) within oral and gut microbial communities. Our RPA assay for mef(A) is highly sensitive (down to picogram levels), and this sensitivity may offer new diagnostic potential. However, the existence of antimicrobial resistance genes within commensal strains of the oral cavity even of healthy individuals raises concerns that a highly sensitive antibiotic-resistance test like ours may detect the genes when no infection is present. However, understanding the dynamics and inter-individual variation even in a healthy resistome is an important part of personalized medicine, which includes the microbiome and associated mediators of antimicrobial resistance. Because the microbiome is a dynamic entity in which antimicrobial resistance genes are shared among members, it is clinically vital to monitor levels of antibiotic resistance genes in commensal bacteria of healthy individuals that may contribute to more severe disease. For example, infections caused by cystic fibrosis are increasingly antibiotic resistant due to the horizontal transfer of resistance genes from commensal bacteria.

To date there is no cheap, easy, rapid assay to measure mef(A) in a patient's healthy microbiome, but we provide such a tool, validated to show the genetic signature correlates with actual erythromycin resistance. Furthermore, having insight into the presence of resistance genes in the (healthy) microbiome of a patient would properly inform clinicians should that person become sick, reducing both morbidity and therapeutic failure and re-treatment. In other words, a patient with intrinsically high levels of mef(A) in her healthy microbiome would be best advised to avoid macrolide treatments if she becomes ill.

The question of whether our RPA assay would distinguish infection from colonization is related to a larger debate in the diagnostic field: when is a molecular assay too sensitive?Molecular detection methods like qPCR or RPA are much more sensitive than culture methods, often identifying many more microbes than culture, leading some to conclude that the diagnostic utility of these methods is limited due to false positives. However, there are several strategies for mitigating this risk: for example, testing only at-risk populations, as applied to testing for C. difficile or Group-A Streptococcus (S. pyogenes). This strategy minimizes the chance of a false-positive detection by not employing the test in cases unlikely to represent true infection. Thus, a clinician might deploy the described mef(A) assay when a patient exhibits symptoms consistent with bacterial infection, to guide choice of therapeutic agent. A second, and more powerful strategy is to focus on levels of the genetic sequence observed. If mef(A) is helping a pathogen cause disease, it will be enriched to a higher copy number than it would be as a sporadic colonizer diluted into a healthy microbial community. By providing quantitative data on relative levels of mef(A), the described RPA assay is ideally suited to this approach, making the determination of an infection a matter of comparing the detected gene level with a threshold (after normalizing to total bacterial load). Mef(A) has been found in a wide variety of bacterial hosts, from Neisseria gonorrhoeae to Enterococcus faecalis and Streptococcus pneumoniae and pyogenes, and it has recently been found within commensal strains including Streptoccous salivarius, as we independently confirmed using RPA. We anticipate the mef(A) assay will become an important tool in the diagnostic toolbox, offering physicians and scientists alike a rapid, accurate measure of macrolide resistance, whether hosted in the upper (S. pyogenes or S. salivarius) or lower respiratory tract (Streptococcus pneumoniae or Staphylococcus aureus or others), or in other regions of the human microbiome.

### Diagnostic Apparatus

In another aspect, a diagnostic apparatus is described consisting of three distinct parts: an array of molecular assays targeting genomic regions of interest, such as the modified RPA assay described above, a robotic system for sample processing and assay setup, and a secure electronic readout with data storage system that interfaces with the customer, the doctor, and the relevant medical records. The described system provides a diagnostic platform technology for detection of pathogens based on genomic DNA/RNA amplification. In one embodiment, the diagnostic station accepts a swab or saliva sample, a robot performs all required nucleic amplification steps in a self-contained unit (eliminating the need for a diagnostic lab), and the results are reported to the patient's doctor (as part of their medical record) and also uploaded onto a smartphone. Robotics are being used to fully automate the setup of the RPA panel, integrating it into a fully CLIA-waved device that can be conveniently provided directly to patients, through minute clinics, or primary care physicians lacking access to certified research laboratories capable of medical diagnostic services. The device will interface remotely with healthcare providers giving them critical-point-of care data which can be used to make antibiotic decisions.

In one aspect, the device will consist of a cabinet containing a robotic arm; the cabinet has an opening in which a patient deposits a raw sample (for example, for respiratory pathogens, a saliva-containing tube). This sample is automatically processed into a raw lysate by brief boiling (3 minutes at 95° C., FIG. 2A) and then diluted into an assay panel plate by the robotic arm. Some of each sample will be saved in a preservative: Zymo Research DNA-RNA shield buffer (catalog # R1100-50), which stabilizes nucleic acids (without refrigeration) for future genomic analysis (contingent upon patient consent). After automated reaction setup the RPA will proceed at 37° C. within an electronic readout system which analyzes the data and presents a report to the patient. In one embodiment, the robotic system will perform self-decontamination between runs. For the first time, we will be able to track the spread of known respiratory pathogens and antibiotic resistance genes in real time. Particularly, as significant numbers of devices are deployed, we will have the potential to predict outbreaks. This data will accumulate over time, providing a goldmine of epidemiological insight, and will likely re-shape our perspective on the spread of respiratory pathogens, providing additional research value to the CDC and other organizations.

In one embodiment, the diagnostic device or kiosk will resemble a small box (approximately the size of a microwave), placed on a counter top or surface with a screen for patient interaction. Inside the kiosk will be the custom-designed robotic system to perform the sterile RPA procedure, consisting of a 3-axis of movement arm implementing pipettes with replaceable tips, a 95° C. heatblock for lysis, a combined 37° C. heatblock and spectrometer readout device, and a software program that interfaces between the robot and detector (hardware) and user-data software (see attachment for CAD images). In addition to the robotic system, the kiosk will provide robot-accessible sterile consumables such as pipet tips, test tube strips, and RPA reagents. Because lyophilization will be used to preserve RPA reagents, they can be supplied at room temperature, avoiding the complication of cooling systems. Therefore, the complete system will instantiate the robotic arm, plastic consumables, lysis heatblock, a heatblock/readout system and biohazard waste disposal bins for liquid and solid waste. Finally, 25% of each sample will be preserved in DNA-RNA shield buffer (catalog # R1100-50) for future de novo pathogen analysis if they are both negative for known pathogens and with patient consent for research. These samples will be stored at room temperature in the DNA-RNA shield buffer (where they are stable for over two years).

In one embodiment, there will be a dispenser of sterile tubes or swabs for on-site sample collection from the patient. All samples taken into the device are liquid-so if taken, a throat or nasal swab will be swirled in the DNA-RNA preservative and lysis buffer, then swab itself removed and disposed in biohazardoust waste. Saliva will be collected simply by spitting into the sample tube with preservative. The liquid-containing sample tube will be deposited into the device through a port that is robotic-arm accessible. To lyse the sample, the system will briefly heat to 95 degrees for 3 minutes to liberate genomic DNA/RNA. The robotic pipette arm will then set up an RPA assay panel, using 75% of the sample, in closed transparent 0.2 ml strip tubes seated in a 37° C. heatblock/readout spectrophotometer. RPA reagents can be used as lyophilized powder pellet, making it ideally suited to robotic setup: the robot has to simply pipet the sample (diluted in to the appropriate volume in RPA buffer) into the tube with pre-lyophilized pellet (which will include primers and probe for the specific assay), add magnesium acetate to activate the reaction, and seal the tubes for the run. Setup directly in the heatblock/readout system minimizes device complexity and optimizes speed and robustness of the workflow, and keeping tubes closed during the amplification minimizes the chance of cross-contamination. Between runs, self-sterilization with UV light will be performed, and the system will automatically re-set for the next sample.

In one embodiment, a respiratory panel will be setup on a 96-well plate (or smaller) with the wells containing the pre-lyophilized pellet, primers and probes for the specific assay targets, a negative control and a bacterial load (positive) control, all in duplicate. To maximize throughput, two different fluorophore readouts: ROX and FAM, will be employed, both of which have been used together effectively in qPCR. Throughput will therefore be optimized by allowing more than one sample to be processed in a single run, and also will have a ‘queuing’ mechanism by which multiple samples can be loaded into the system for sequential automated processing. This will facilitate, for example, a nurse practitioner that wants to load the system with many samples overnight and let them work through the device after she leaves for the day.

In one embodiment, the assays are based on Recombinase Polymerase Assay, a relative of Polymerase Chain Reaction (PCR) but not requiring temperature cycling. The described modified RPA assay was based on ‘RPA Nfo’ kit (TwistDx, UK). The ‘RPA Nfo’ kit uses the nfo enzyme (Endonuclease IV) that cleaves an abasic site in the probe to promote secondary amplification. As described above, we designed a functioning assay to detect a common antibiotic resistance gene (Macrolide Efflux A, or mef(A)) using the described unique primers and the inventive Taq-Man-like probe. This constitutes a proof-of-concept that any genomic region can in principle be targeted by primer and probe design and optimization, as described herein.

The RPA probe is not, by default, set up to use Taq-Man style detection: TwistDx recommends using lateral flow dipsticks as a readout (Milenia Hybridetect flow strips) but can be detected by SYBR green or by gel electrophoresis. Our described probe used a quencher on the probe to invent a hydrolysis-style ‘RPA-Man’ system in which the cleavage by nfo/Endonuclease IV enzyme, rather than the 3′-5′ exonuclease activity of the polymerase (as in Taq-Man) separates the fluorophore from a quencher and activates the signal. In one embodiment, 5′ FAM fluorophore and 3′ Iowa Black quencher were used, but any suitable fluorophore/quencher combination can be used.

In one embodiment, the robotic system is generally designed to take in a raw sample, briefly boil it (heat to 95° C. for about 3 min) to lyse the bacteria and liberate genomic DNA, and then perform the RPA assay. Liberated RNA is then converted to cDNA in a reverse transcription step. RPA is relatively impervious to contaminants in the solution and is extremely sensitive to low amounts of DNA/cDNA. After setup, the RPA will proceed at about 37° C. within an electronic readout system. In one embodiment, the robotic system also includes a method of self-decontamination between runs. In various embodiments, the system comprises a robotic arm or conveyor belt. In one embodiment, for the privacy of the patient, the system is provided in a larger enclosure akin to a photo booth for sample collection. This enclosure would also comprise various consumables such as a dispenser of sterile tubes and swabs for on-site sample collection.

The system will also include an electronic readout and data system. In one embodiment, a qPCR-style electric readout is employed, allowing amplification in real-time to be monitored. This will produce qPCR-like amplification curves that yield quantitative information on pathogen levels in the sample. In one embodiment, the system also comprises software to perform at least one of reading the data, calculate amplification curves, build reports for consumer and doctor, and automatically email the results to both. In one embodiment, the customer would then receive a PDF report within 20-30 minutes of depositing the sample. In an additional embodiment, the report will also be delivered to the consumer's smartphone as well as their email.

The described system would provide extreme ease of use: the user simply deposits a swab or tube of fluid he/she wishes to test; robotics would handle the sample and results would be reported electronically to the patient and also interface with the healthcare system through patient portal electronic medical records (FIG. 10). The patient's physician would be notified, if available, or other medical professionals specified by the patient (FIG. 10), thus ‘closing the loop’ and enabling informed medical decisions by the doctor. In various embodiments, the system provides extremely fast results: the diagnostic report would be generated within 1 hour. By leveraging genomic information inherent in an infectious agent, our approach yields unprecedented sensitivity and specificity as has been reported with qPCR methods albeit not implemented optimally as outlined above. Finally, in various embodiments, the innovative robotic implementation will be able to handle swabs (dry or wet) as well as liquid. Buccal swabs, spit samples, or even dry swabs of medical equipment-allowing the tracking of hospital-acquired infectious agents-will be straightforward.

In various embodiments, the described system, also referred to as MicroInvestigate (MI) Stations, are an automated, pre-point-of-care technology, preliminary screening test composed of robotically integrated customized with viral-vs-bacterial assay panels with novel nucleic-acid based technology. In one embodiment, the system is composed of three key parts: (1) DNA extraction and Recombinase Polymerase Amplification (RPA), (2) integrated robotics and software, and (3) booth for sample collection.

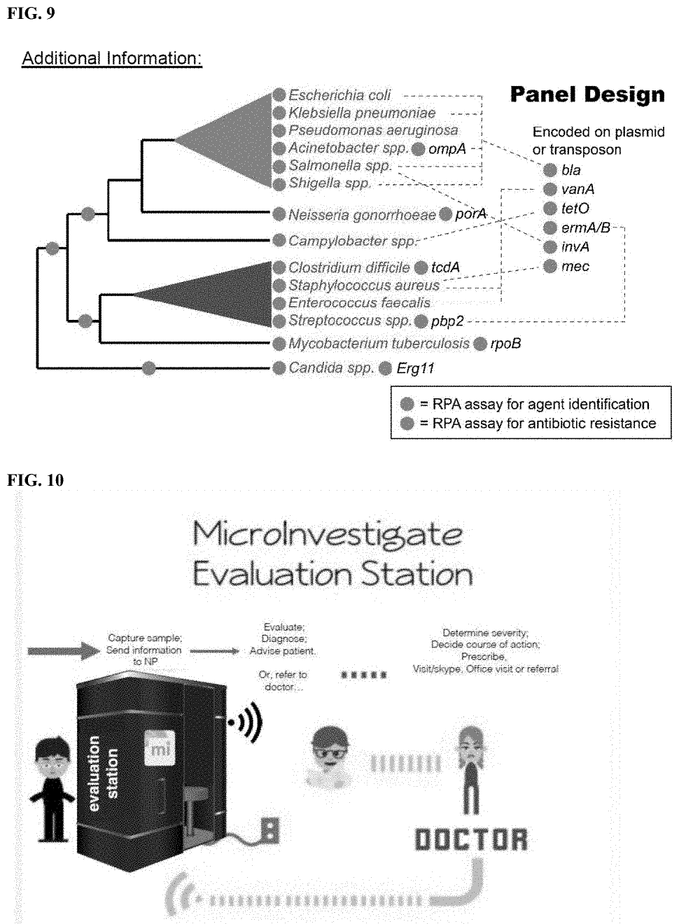

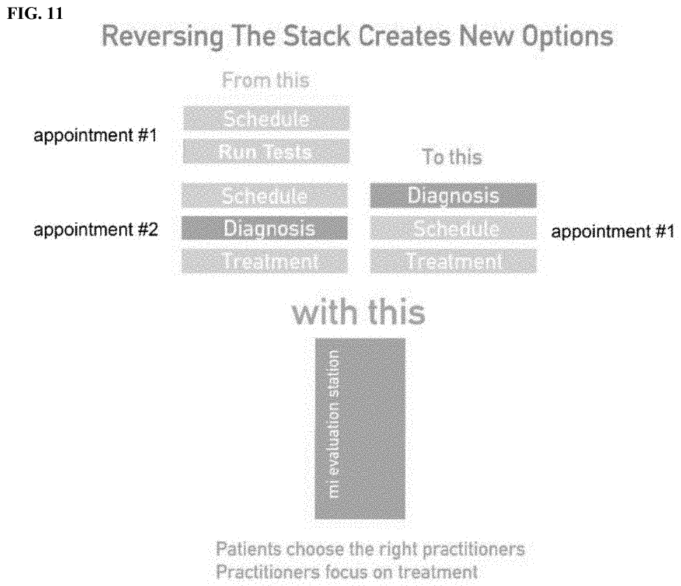

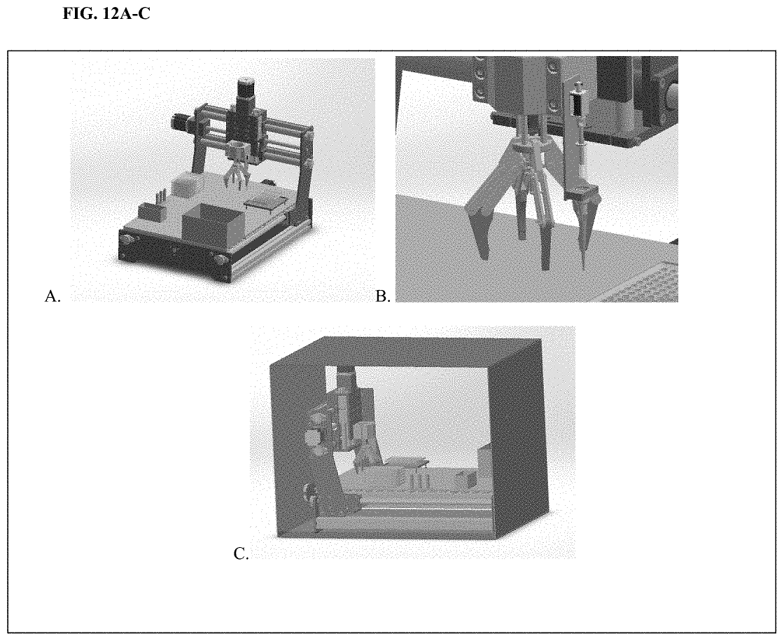

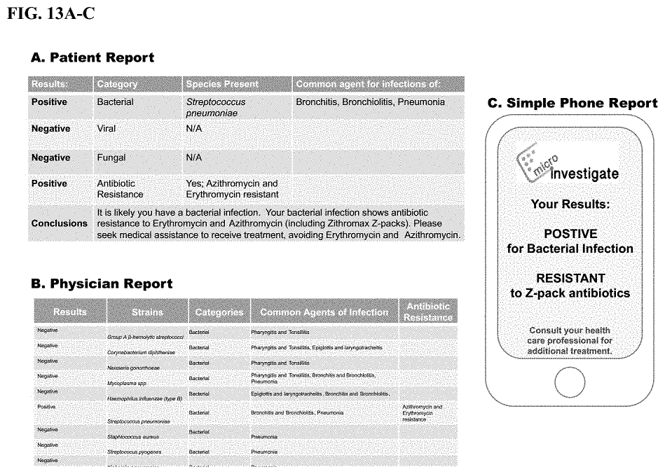

In one aspect, our solution uses RPA which is currently only being used to detect plant pathogens. RPA is a nucleic-acid based assay that slightly differs from PCR/qPCR, however, the overall concept is the same. Nucleic-acid based testing is highly sensitive and specialized and thus, accurately detects infections. DNA amplification is crucial to most nucleic acid testing strategies, but requires expensive equipment and labor-intensive experimental procedures. RPA differs from standard PCR/qPCR by coupling isothermal recombinase-driven primer targeting of template material with strand-displacement DNA synthesis. Therefore, it accomplishes exponential amplification without the need for pretreatment of sample DNA. The reactions operate at constant low temperature and are specific and rapid. Using a probe-based detection system, the combined RPA amplification/detection process has been demonstrated by a testing for the pathogen methicillin-resistant Staphylococcus aureus. Additionally, this method has demonstrated to be sensitive to less than ten copies of genomic DNA. Moreover, this technique provides real-time results in 30 minutes or less. In combination with the RPA, our test contains a panel composed of viral-vs-bacterial discrimination, species identification, and primers designed to amplify antibiotic resistance genes within bacterial species (Table 1). These panels are designed with a nested evolutionary scope (see FIG. 9). Thus, some assays will determine broad groupings of bacterial species, while other assays will narrow the scope down to genus or even species level. Our assays will rule out species, not just rule them in, powerfully narrowing the diagnostic field and simplifying the diagnostic task. In various embodiments, assays for antibiotic resistance will also be included, which does not necessarily track with bacterial lineage (i.e., can be transferred among species on plasmids or other mobile genetic elements). In this way, a ‘universal bacterial detector’ test is provided that supersedes all tests on the market, and at lower market price.

The second component of the described system, integrated robotics and software, are essential for automating, processing and evaluating the results. Due to the simplicity of RPA, the main use of the robotics in the system will be DNA extraction from the sample and then transfer to the heating block for testing. In various embodiments, the software will play a major role in (1) collecting, (2) analyzing and (3) reporting results. According to one embodiment, a task flowchart is as follows: Step (1), a patient will log-on to our system and be provided with step-by-step instructions on how to collect a sample. Step (2), after running the described test, the software programing will analyze the results of the test. Step (3), two reports, a patient-friendly report and a doctor-friendly report, are recorded and distributed. In one embodiment, the patient's report will be sent to an allocated email address and will also have the option to send a doctor's report to their primary care physician or a local physician to set up an appointment. Moreover, in further embodiments, the system will interface with patient portal software, allowing patients to save their test results and in some cases, to get diagnosed and prescription (if necessary) from an in-network nurse practitioner. Thus, as one potential benefit of the described system, the prevention of many patients from even needing to see a doctor, or making a single visit rather than multiple. Lastly, according to one embodiment, the described system is designed similarly to a photo-booth/kiosk. This will provide privacy for sample collection and the enclosed-environment will help to reduce additional contamination. The automated kiosk design will reduce cross-contamination between samples while also providing privacy and space for the patient to take the sample. We note that healthcare kiosks (such as higi and Pursuant Health) are already in place in many grocery stores and other public spaces, where they provide blood pressure or other simple assays. The described system would thus fit well in the current market landscape and should be readily accepted by customers. Locating these kiosks in high-traffic areas should drive significant usage by patients who need rapid diagnostic answers to their questions: particularly parents of young children, or college-age students, who have little time to schedule doctor's appointments or to cancel work.

The described kiosk will implement RPA within a user-friendly system—a sample drop-and-go interface implementation of state-of-the art molecular testing. Once the run completes and electronic readout system analyzes the data, the patient will be provided with a report. The robotic system will be designed to self-clean between runs and autonomously reset itself, so the maintenance of the device is limited to periodic restocking of supplies and removing biohazard waste for disposal. Standard qPCR results are not user-friendly for the everyday consumer. Therefore, a simplified version of the results, the MiKi Report, will be sent direct to the consumer. It is critical that it be informative, concise, and clear (FIG. 10A). The device will also generate a more detailed report for the physician (FIG. 10B), to be provided to the patient also so they can bring it to a healthcare appointment. An advanced version of the product will also provide a smartphone report containing the minimum amount of actionable data (FIG. 10C). Running the device will be extremely easy: the MiKi will encompass a secure system that allows the user to create an account or to log in as a guest, fill out a symptom questionnaire, pay for the test, and deposit the sample; the user will then be automatically provided their reports by email once the run is complete.

References, each of which is incorporated herein in its entirety:
1. National Institutes of Health: NIAID's Antibacterial Resistance Program: Current Status and Future Directions. https://wwwniaidnihgov/sites/default/files/arstrategicplan2014pdf.
2. CDC: Antibiotic/Antimicrobial Resistance. https://wwwcdcgov/drugresistance/indexhtml.
3. World Health Organization Global Action Plan on Antimicrobial Resistance. http://wwwwprowhoint/entity/drug_resistance/resources/global_action_plan_engpdf.
4. Editors P M: Antimicrobial Resistance: Is the World UNprepared? PLoSMed 2016, 13(9):e1002130.
5. Obama B: Executive Order—Combating Antibiotic-Resistant Bacteria. https://obamawhitehousearchivesgov/the-press-office/2014/09/18/executive-order-combating-antibiotic-resistant-bacteria 2014.
6. National Action Plan for Combating Antibiotic Resistant Bacteria. In.
7. Caliendo A M, Gilbert D N, Ginocchio C C, Hanson K E, May L, Quinn T C, Tenover F C, Alland D, Blaschke A J, Bonomo R A et al: Better Tests, Better Care: Improved Diagnostics for Infectious Diseases. Clin Infect Dis 2013, 57: S 139-S 170.
8. de Kraker M E, Stewardson A J, Harbarth S: Will 10 Million People Die a Year due to Antimicrobial Resistance by 2050? PLoS Med 2016, 13(11):e1002184.
9. Piepenburg O, Williams C H, Stemple D L, Armes N A: DNA detection using recombination proteins. Plos Biol 2006, 4(7):1115-1121.
10. Holland P M, Abramson R D, Watson R, Gelfand D H: Detection of Specific Polymerase Chain-Reaction Product by Utilizing the 5′-]3′ Exonuclease Activity of Thermus-Aquaticus DNA-Polymerase. P Natl Acad Sci USA 1991, 88(16):7276-7280.
11. Levin J D, Johnson A W, Demple B: Homogeneous Escherichia-Coli Endonuclease-Iv—Characterization of an Enzyme That Recognizes Oxidative Damage in DNA. J Biol Chem 1988, 263(17):8066-8071.
12. Lei R, Yan Z Y, Hu F, Zhu S F, Xiong Y F, Fan X H: Rapid identification of quarantine invasive Solanum elaeagnifolium by real-time, isothermal recombinase polymerase amplification assay. Rsc Adv 2017, 7(83):52573-52580.
13. Patel P, Abd El Wahed A, Faye O, Pruger P, Kaiser M, Thaloengsok S, Ubol S, Sakuntabhai A, Leparc-Goffart I, Hufert F T et al: A Field-Deployable Reverse Transcription Recombinase Polymerase Amplification Assay for Rapid Detection of the Chikungunya Virus. PLoS Negl Trop Dis 2016, 10(9):e0004953.
14. Sun K, Xing W, Yu X, Fu W, Wang Y, Zou M, Luo Z, Xu D: Recombinase polymerase amplification combined with a lateral flow dipstick for rapid and visual detection of Schistosoma japonicum. Parasit Vectors 2016, 9:476.
15. Chao C C, Belinskaya T, Zhang Z, Ching W M: Development of Recombinase Polymerase Amplification Assays for Detection of Orientia tsutsugamushi or Rickettsia typhi. PLoS Negl Trop Dis 2015, 9(7):e0003884.
16. Crannell Z, Castellanos-Gonzalez A, Nair G, Mejia R, White A C, Richards-Kortum R: Multiplexed Recombinase Polymerase Amplification Assay To Detect Intestinal Protozoa. Anal Chem 2016, 88(3): 1610-1616.
17. Crannell Z A, Castellanos-Gonzalez A, Irani A, Rohrman B, White A C, Richards-Kortum R: Nucleic Acid Test to Diagnose Cryptosporidiosis: Lab Assessment in Animal and Patient Specimens. Analytical Chemistry 2014, 86(5):2565-2571.
18. Boyle D S, Lehman D A, Lillis L, Peterson D, Singhal M, Armes N, Parker M, Piepenburg O, Overbaugh J: Rapid Detection of HIV-1 Proviral DNA for Early Infant Diagnosis Using Recombinase Polymerase Amplification. Mbio 2013, 4(2).
19. Natoli M E, Rohrman B A, De Santiago C, van Zyl G U, Richards-Kortum R R: Paper-based detection of HIV-1 drug resistance using isothermal amplification and an oligonucleotide ligation assay. Analytical Biochemistry 2018, 544:64-71.
20. Escadafal C, Paweska J T, Grobbelaar A, le Roux C, Bouloy M, Patel P, Teichmann A, Donoso-Mantke O, Niedrig M: International External Quality Assessment of Molecular Detection of Rift Valley Fever Virus. Plos Neglect Trop D 2013, 7(5).
21. Euler M, Wang Y, Heidenreich D, Patel P, Strohmeier O, Hakenberg S, Niedrig M, Hufert F T, Weidmann M: Development of a panel of recombinase polymerase amplification assays for detection of biothreat agents. J Clin Microbiol 2013, 51(4):1110-1117.
22. Abd El Wahed A, Patel P, Heidenreich D, Hufert F T, Weidmann M: Reverse transcription recombinase polymerase amplification assay for the detection of middle East respiratory syndrome coronavirus. PLoS Curr 2013, 5.
23. Abd El Wahed A, El-Deeb A, El-Tholoth M, Abd El Kader H, Ahmed A, Hassan S, Hoffmann B, Haas B, Shalaby M A, Hufert F T et al: A portable reverse transcription recombinase polymerase amplification assay for rapid detection of foot-and-mouth disease virus. PLoS One 2013, 8(8):e71642.
24. Amer H M, Abd El Wahed A, Shalaby M A, Almajhdi F N, Hufert F T, Weidmann M: A new approach for diagnosis of bovine coronavirus using a reverse transcription recombinase polymerase amplification assay. J Virol Methods 2013, 193(2):337-340.
25. Bonney L C, Watson R J, Afrough B, Mullojonova M, Dzhuraeva V, Tishkova F, Hewson R: A recombinase polymerase amplification assay for rapid detection of Crimean-Congo Haemorrhagic fever Virus infection. PLoS Negl Trop Dis 2017, 11(10):e0006013.
26. Mo Y J, Cui F, Li D R, Dai Y, Li X M, Zhang X Y, Qiu Y L, Yin Y B, Zhang X M, Xu W C: Establishment of a rapid and sensitive method based on recombinase polymerase amplification to detect mts90, a new molecular target of Mycobacterium tuberculosis. Rsc Adv 2017, 7(79):49895-49902.
27. Ng B Y C, Wee E J H, Woods K, Anderson W, Antaw F, Tsang H Z H, West N P, Trau M: Isothermal Point Mutation Detection: Toward a First-Pass Screening Strategy for Multidrug-Resistant Tuberculosis. Anal Chem 2017, 89(17):9017-9022.
28. Kersting S, Rausch V, Bier F F, von Nickisch-Rosenegk M: Multiplex isothermal solid-phase recombinase polymerase amplification for the specific and fast DNA-based detection of three bacterial pathogens. Mikrochim Acta 2014, 181(13-14):1715-1723.
29. Krolov K, Frolova J, Tudoran O, Suhorutsenko J, Lehto T, Sibul H, Mager I, Laanpere M, Tulp I, Langell O: Sensitive and Rapid Detection of Chlamydia trachomatis by Recombinase Polymerase Amplification Directly from Urine Samples. J Mol Diagn 2014, 16(1): 127-135.
30. Euler M, Wang Y, Otto P, Tomaso H, Escudero R, Anda P, Hufert F T, Weidmann M: Recombinase polymerase amplification assay for rapid detection of Francisella tularensis. J Clin Microbiol 2012, 50(7):2234-2238.
31. Daher R K, Stewart G, Boissinot M, Bergeron M G: Isothermal recombinase polymerase amplification assay applied to the detection of group B streptococci in vaginal/anal samples. Clin Chem 2014, 60(4):660-666.
32. Sutcliffe J, Tait-Kamradt A, Wondrack L: Streptococcus pneumoniae and Streptococcus pyogenes resistant to macrolides but sensitive to clindamycin: a common resistance pattern mediated by an efflux system. Antimicrob Agents Chemother 1996, 40(8):1817-1824.
33. Clancy J, Petitpas J, Dib-Hajj F, Yuan W, Cronan M, Kamath A V, Bergeron J, Retsema J A: Molecular cloning and functional analysis of a novel macrolide-resistance determinant, mefA, from Streptococcus pyogenes. Mol Microbiol 1996, 22(5):867-879.
34. Banks D J, Porcella S F, Barbian K D, Martin J M, Musser J M: Structure and distribution of an unusual chimeric genetic element encoding macrolide resistance in phylogenetically diverse clones of group A Streptococcus. J Infect Dis 2003, 188(12):1898-1908.
35. Giovanetti E, Brenciani A, Vecchi M, Manzin A, Varaldo P E: Prophage association of mef(A) elements encoding efflux-mediated erythromycin resistance in Streptococcus pyogenes. J Antimicrob Chemother 2005, 55(4):445-451.
36. Klaassen C H, Mouton J W: Molecular detection of the macrolide efflux gene: to discriminate or not to discriminate between mef(A) and mef(E). Antimicrob Agents Chemother 2005, 49(4):1271-1278.
37. Chaffanel F, Charron-Bourgoin F, Libante V, Leblond-Bourget N, Payot S: Resistance Genes and Genetic Elements Associated with Antibiotic Resistance in Clinical and Commensal Isolates of Streptococcus salivarius. Appl Environ Microbiol 2015, 81(12):4155-4163.
38. Banks D J, Porcella S F, Barbian K D, Beres S B, Philips L E, Voyich J M, DeLeo F R, Martin J M, Somerville G A, Musser J M: Progress toward characterization of the group A Streptococcus metagenome: complete genome sequence of a macrolide-resistant serotype M6 strain. J Infect Dis 2004, 190(4):727-738.
39. Curran T, Coyle P V, McManus T E, Kidney J, Coulter W A: Evaluation of real-time PCR for the detection and quantification of bacteria in chronic obstructive pulmonary disease. FEMS Immunol Med Microbiol 2007, 50(1): 112-118.
40. Green N M, Zhang S, Porcella S F, Nagiec M J, Barbian K D, Beres S B, LeFebvre R B,
Musser J M: Genome sequence of a serotype M28 strain of group a streptococcus: potential new insights into puerperal sepsis and bacterial disease specificity. J Infect Dis 2005, 192(5):760-770.
41. Weisblum B: Erythromycin resistance by ribosome modification. Antimicrob Agents Chemother 1995, 39(3):577-585.
42. Bemer-Melchior P, Juvin M E, Tassin S, Bryskier A, Schito G C, Drugeon H B: In vitro activity of the new ketolide telithromycin compared with those of macrolides against Streptococcus pyogenes: Influences of resistance mechanisms and methodological factors. Antimicrob Agents Ch 2000, 44(11):2999-3002.
43. Leclercq R, Courvalin P: Resistance to macrolides and related antibiotics in Streptococcus pneumoniae. Antimicrob Agents Ch 2002, 46(9):2727-2734.
44. Lean W L, Amup S, Danchin M, Steer A C: Rapid diagnostic tests for group A streptococcal pharyngitis: a meta-analysis. Pediatrics 2014, 134(4):771-781.
45. Stewart E H, Davis B, Clemans-Taylor B L, Littenberg B, Estrada C A, Centor R M: Rapid antigen group A streptococcus test to diagnose pharyngitis: a systematic review and meta-analysis. PLoS One 2014, 9(11):el 11727.
46. Baron S: Medical microbiology, 4th edn. Galveston, Tex.: University of Texas Medical Branch at Galveston; 1996.
47. Reimer L G, Carroll K C: Role of the microbiology laboratory in the diagnosis of lower respiratory tract infections. Clin Infect Dis 1998, 26(3):742-748.
48. Mu X Q, Nakano R, Nakano A, Ubagai T, Kikuchi-Ueda T, Tansho-Nagakawa S, Kikuchi H, Kamoshida G, Endo S, Yano H et al: Loop-mediated isothermal amplification: Rapid and sensitive detection of the antibiotic resistance gene ISAba1-bla(OXA-51-like) in Acinetobacter baumannii. J Microbiol Meth 2016, 121:36-40.
49. Kim H J, Kim H S, Lee J M, Yoon S S, Yong D: Rapid detection of Pseudomonas aeruginosa and Acinetobacter baumannii Harboring bla(VIM-2), bla(IMP-1) and bla(OXA-23) genes by using loop-mediated isothermal amplification methods. Ann Lab Med 2016, 36(1): 15-22.
50. Stedtfeld R D, Stedtfeld T M, Waseem H, Fitschen-Brown M, Guo X, Chai B, Williams M R, Shook T, Logan A, Graham A et al: Isothermal assay targeting class 1 integrase gene for environmental surveillance of antibiotic resistance markers. J Environ Manage 2017, 198(Pt 1):213-220.
51. Mu X Q, Liu B B, Hui E, Huang W, Yao L C, Duo L B, Sun W Y, Li G Q, Wang F X, Liu S L: A rapid loop-mediated isothermal amplification (LAMP) method for detection of the macrolide-streptogramin type B resistance gene msrA in Staphylococcus aureus. J Glob Antimicrob Re 2016, 7:53-58.
52. Imirzalioglu C, Falgenhauer L, Schmiedel J, Waezsada S E, Gwozdzinski K, Roschanski N, Roesler U, Kreienbrock L, Schiffmann A P, Irrgang A et al: Evaluation of a Loop-Mediated Isothermal Amplification-Based Assay for the Rapid Detection of Plasmid-Encoded Colistin Resistance Gene mcr-1 in Enterobacteriaceae Isolates. Antimicrob Agents Ch 2017, 61(4).
53. Hu C, Kalsi S, Zeimpekis I, Sun K, Ashburn P, Turner C, Sutton J M, Morgan H: Ultra-fast electronic detection of antimicrobial resistance genes using isothermal amplification and Thin Film Transistor sensors. Biosens Bioelectron 2017, 96:281-287.
54. Dixon J M, Miller D C: Value of dilute inocula in cultural examination of sputum. Lancet 1965, 2(7421):1046-1048.
55. Cousin S, Jr., Whittington W L, Roberts M C: Acquired macrolide resistance genes in pathogenic Neisseria spp. isolated between 1940 and 1987. Antimicrob Agents Chemother 2003, 47(12):3877-3880.
56. Luna V A, Coates P, Eady E A, Cove J H, Nguyen T T, Roberts M C: A variety of gram-positive bacteria carry mobile mef genes. J Antimicrob Chemother 1999, 44(1):19-25.
57. Luna V A, Heiken M, Judge K, Ulep C, Van Kirk N, Luis H, Bemardo M, Leitao J, Roberts M C: Distribution of mef(A) in gram-positive bacteria from healthy Portuguese children. Antimicrob Agents Chemother 2002, 46(8):2513-2517.

Applicants incorporate by reference the material contained in the accompanying computer readable Sequence Listing identified as 023783.61_ST25.txt, having a file creation date of Dec. 3, 2019 at 11:39 A.M. and file size of 27.2 KB.

What is claimed is:

1. A system for the detection of pathogens and/or antimicrobial resistance in a sample, the system comprising
a detection module, and
a data analysis and processing module,
wherein the detection module comprises a fluid handling system for performing Recombinase Polymerase Assay (RPA) on the sample.

2. The system of claim 1, wherein the fluid handling system comprises means for accepting the sample, subjecting the sample to lysis conditions to release genomic DNA or RNA, produce cDNA from the RNA, and performing RPA on the released genomic DNA or produced cDNA.

3. The system of claim 2, wherein performing RPA comprises contacting the DNA/cDNA with at least one primer and an analyte-specific probe under conditions sufficient to result in the detection of pathogen and/or antimicrobial resistance in the sample.

4. The system of claim 3, wherein the analyte-specific probe comprises an oligonucleotide substantially complementary to a target sequence of the analyte, a reporter dye covalently attached to one end of the oligonucleotide and a quencher attached to the other end of the oligonucleotide, where the oligonucleotide further comprises an internal abasic site, wherein the analyte-specific probe uses the quencher as a 3′ blocking moiety, and the abasic site interfaces with a nuclease during amplification cycles.

5. The system of claim 1, wherein the data analysis and processing module compiles the data from the RPA assay and provides a readout to the consumer of whether the analyte is detected in the sample.

6. The system of claim 1, wherein the pathogen and/or microbial resistance that is detected is selected from the group consisting of Chlamydia spp., Corynebacterium diphtheria, Neisseria gonorrhoeae, Mycoplasma pneumoniae, Haemophilus influenza, Streptococcus pneumoniae, Staphylococcus aureus, Streptococcus pyogenes (Group A strep), Klebsiella pneumoniae, E. Coli, Candida albicans, Histoplasma capsulatum, Adenovirus, Coxsackieviruses, Influenza A and B, Parainfluenza viruses, Rhinovirus, Coronavirus, Epstein-Barr Virus, Cytomegalovirus, Herpes simplex virus, Respiratory syncytial virus, Hantavirus, mecA, mef(A), ermB, KPC, NDM-1, OXA, bla_{CTX-M}, AmpC, tetO, vanA, tetM, ftsl, mprF, mcr-1, qnrA, gyrA, gyrB, parE, parC, rpoB, and cfr.

7. The system of claim 6, wherein the pathogen and/or microbial resistance is detected using at least one of the following primer pair sets:

| | |
|---|---|
| ErmB_F1 | ATTCACCAAGATATTCTACAGTTTCAATTC |
| ErmB_F2 | TTGAATTAGACAGTCATCTATTCAACTTATC |
| ErmB_R1 | CACTGTTTACTTTTGGTTTAGGATGAAAGC |
| ErmB_R2 | CCAATATTTATCTGGAACATCTGTGGTATG |
| mecA_F1 | ATGGAGTTGAAAGATTTCTTGATTCCTCAAG |
| mecA_F2 | CAATTAGTATGGACGATTTAGAAGAAAGAGG |
| mecA_R1 | ACATATCACCAAACTCTGCTAAATCTTCAAG |
| mecA_R2 | TCCAATTTTTCATGAGCTTTGACATCTCCC |
| AmpC_F1 | TGAGCTAGGATCGGTTAGTAAGACGTTTAAC |
| AmpC_F2 | TATTATTTCACCTGGGGTAAAGCCGATATCG |
| AmpC_R1 | ATGCAGTAATGCGGCTTTATCCCTAACGTC |
| AmpC_R2 | GTCTGGTCATTGCCTCTTCGTAACTCATTC |
| blaCTX-M_F1 | GACGTACAGCAAAAACTTGCCGAATTAGAG |
| blaCTX-M_F2 | AGGCAGACTGGGTGTGGCATTGATTAACAC |
| blaCTX-M_R1 | TCGCTGATTTAACAGATTCGGTTCGCTTTC |
| blaCTX-M_R2 | CCGCAATCGGATTATAGTTAACAAGGTCAG |
| KPC_F1 | GATACCGGCTCAGGCGCAACTGTAAGTTAC |
| KPC_F2 | TCGCTAAACTCGAACAGGACTTTGGCGGCT |
| KPC_R1 | CATCCGTTACGGCAAAAATGCGCTGGTTCC |
| KPC_R2 | CAAATTGGCGGCGGCGTTATCACTGTATTG |
| NDM-1_F1 | GATCCCAACGGTGATATTGTCACTGGTGTG |
| NDM-1_F2 | TAGTGCTCAGTGTCGGCATCACCGAGATTG |
| NDM-1_R1 | CGACTTATGCCAATGCGTTGTCGAACCAGC |
| NDM-1_R2 | CAGATCCTCAACTGGATCAAGCAGGAGATC |
| OXA_F1 | GAATGGAGATCTGGAACAGCAATCATACAC |
| OXA_F2 | TCGCATTATCACTTATGGCATTTGATGCGG |
| OXA_R1 | CCATGCTTCTGTTAATCCGTTGTTTCTTTC |
| OXA_R2 | CCAGAGAAGTCTTGATTTCCATAATCAAAATC |
| tetO_F1 | GACAGATACAATGAATTTGGAGCGTCAAAG |
| tetO_F2 | GTTTATTGTATACCAGTGGTGCAATTGCAG |
| tetO_R1 | CATTATCTGTAGTGCATGAAACAGTATACGG |
| tetO_R2 | CCATCCTTTGCAGAAACTAATAATACTGCTC |
| vanA_F1 | CGAATTGGACTACGCAATTGAATCGGCAAG |
| vanA_F2 | TTAATTGAGCAGGCTGTTTCGGGCTGTGAG |
| vanA_R1 | GTAAAAACATATCCACACGGGCTAGACCTC |
| vanA_R2 | CTGCGGGAACGGTTATAACTGCGTTTTCAG |
| TetM_F1 | CGCTTCTACGATATTACGTGGATTCTACGA |
| TetM_F2 | GTGCACTGTTGCAAGAAAAGTATCATGTGG |
| TetM_R1 | GATTGATTTAAGTATCCAAGAGAAACCGAGC |
| TetM_R2 | CAGGGCTATAGTATAAGCCATACTTAAAACAG |
| ftsl_F1 | GGTCAAATACTACAGATGCAACTTAAACGG |
| ftsl_F2 | GCTTTTTACTTTTCCATTGCTGTAACCACTC |
| ftsl_R1 | TCGGTCTTCTTTTCCTCTATCTGCGCATTG |
| ftsl_R2 | GCAATTTCTTTCAAACGTTCTGCACGTAATAG |
| mprF_F1 | CATTGCTAATTGTATTCCATGTTTTCGATGC |
| mprF_F2 | GGCGTTAGAGCAATGGTTTATAAAAACTATAC |
| mprF_R1 | GAATAAAGCTGACTAAACCTGATAATGCAG |
| mprF_R2 | CCGGTACAAAATAGTACGCAAAACGATATA |
| mcr-1_F1 | GATAAAATCAGCCAAACCTATCCCATCGCG |
| mcr-1_F2 | CGCTATGTGCTAAAGCCTGTGTTGATTTTG |
| mcr-1_R1 | CGCATGATAAACGCTGCGTTTAATAGATCC |
| mcr-1_R2 | CCTTAACAAAAGCCACAAGCAAACTTGGTA |
| QnrA_F1 | GCTTTTATCAGTGTGACTTCAGCCACTGTC |
| QnrA_F2 | CAGCAAGAGGATTTCTCACGCCAGGATTTG |
| QnrA_R1 | CATTGCTCCAGTTGTTTTCAAACAGCTCGC |
| QnrA_R2 | CAGAAGTACATCTTATGGCTGACTTGATTG |
| gyrA_F1 | GTGATCACCGAGTTGCCGTATCAGGTCAAC |
| gyrA_F2 | CAAGCTGGCCGGCATTTCCAACATTGAGGA |
| gyrA_R1 | GGTCAACGTAATAGCGGATCAGCTGGTCCA |
| gyrA_R2 | GTCTGCAGCTGGGTGTGCTTGTAAAGGTTAT |
| gyrB_F1 | GTGTGAAGGGCTTCGTCGAGTACATCAACA |
| gyrB_F2 | CACGGTCGAATATCACTACGACATCCTCGC |
| gyrB_R1 | GATGTACTTGTTGATGACGCGCGTCATCGC |
| gyrB_R2 | CAGAAGAAACCAGCTTGTCCTTCGTCTGCG |
| parE_F1 | GTTGAAATCACTCGCGATGGTGCAATCTAC |
| parE_F2 | GTACAGCACCCAAGTCTAAAACAGGTACCA |
| parE_R1 | CTTCCACTTGGAAACCATTATCTTCTCCTT |
| parE_R2 | GGTCTCCTTGTCTTCGTTAAGATAAGAAAC |
| parC_F1 | CTATCTATGATGCCATGGTTCGTATGTCAC |
| parC_F2 | GGTAACATCATGGGGAATTTCCACCCACAC |
| parC_R1 | GCAATCTCAGACAAACGCGCCTCAGTATAA |
| parC_R2 | CAGTCGAACCATTGACCAAGAGGTTTGGAA |
| rpoB_F1 | GTACTTCGACGAGACCATTGACAAGTCCAC |
| rpoB_F2 | GATGATGACCGAGAAGGGCACGTTCATCAT |
| rpoB_R1 | GAACAAGTTTTCCAACAGCGTCTGCGCTGA |
| rpoB_R2 | CAGCCCGAGCTTCTTGTTGACCTTATAGCG |
| cfr_F1 | GAAGTATCAAAGAATGAGAGAGTAGAAACG |
| cfr_F2 | GGATATGAAGGTTCTTCCAAAATTACTTAG |
| cfr_R1 | AGAGCTTCACCCATTCCCATAAAAGAAATG |
| cfr_R2 | GGAAGTATAAAACTTGATCTGTTATCTCATC |
| MefA_F1 | GCGGTTACGCCACTTTTAGTACCAGAAGAACAGCT |
| FefA_R1 | TTTAGTTCCCAAACGGAGTATAAGAGTGCTGCAAC. |

8. The system of claim 6, wherein the pathogen and/or microbial resistance is detected using at least one analyte-specific probe selected from the group consisting of:
a) ErmB_quench_probe
/5HEX/TACCTTGGATATTCACCGAACACTAGGGTTG/idSp/
TCTTGCACACTCAAG/3IABkFQ/,
b) mecA_quench_probe
/5Cy3/TTTAAAGATAGTGGTATGCTTAGTTTTCGA/idSp/
TGACTCCACGCAAGG/3IABkRQ/,
c) AmpC_quench_probe
/5Cy5/TGGCCAGAACTGACAGGCAAACAGTGGCAG/idSp/
GTATCCGCCTGCTGC/3IABkRQ/,
d) blaCTX-M_quench_probe
/56-
FAM/TTCGCAAATACTTTATCGTGCTGATGAGCG/idSp/
TTTGCGATGTGCAGC/3IABkFQ/,
e) KPC_quench_probe
/5HEX/TGCTGCCGCTGTGCTGGCTCGCAGCCAGCA/idSp/
CAGGCCGGCTTGCTG/3IABkFQ/,
f) NDM-1_quench_probe
/5Cy3/TTGCTGGTTCGACCCAGCCATTGGCGGCGA/idSp/
AGTCAGGCTGTGTTG/3IABkRQ/,
g) OXA_quench_probe
/5Cy5/TTGGGTTTCGCAAGAAATAACCCAAAAAAT/idSp/
GGATTAAATAAAATC/3IABkRQ/,
h) tetO_quench_probe
/56-
FAM/TGGGAGGATGTAAAAGTCAACATTATAGAT/idSp/
CGCCAGGCCATATGG/3IABkFQ/,
i) vanA_quench _probe
/5HEX/TCAGGCTGCAGTACGGAATCTTTCGTATTC/idSp/
TCAGGAAGTCGAGCC/3IABkFQ/,
j) TetM_quench_probe
/5HEX/TGCCGCCAAATCCTTTCTGGGCTTCCATTG/idSp/
TTTATCTGTATCACC/3IABkFQ/,
k) ftsl_quench_probe
/5Cy3/TATTATTTTTATGCAGACCAAGCTCTTGCA/idSp/
GTGCAGAATGATTTG/3IABkRQ/,
l) mprF_quench_probe
/5Cy5/TGTGTTGAATGGTTAGCAGCTGCAGTTGTA/idSp/
TATATTTCTGTGGTG/3IABkRQ/,
m) Mcr-1_quench_probe
/56-FAM/TATTTTACTGACACTTATGGCACGGTCTAT/idSp/
ATACGACCATGCTCC/3IABkFQ/,
n) Qnr_quench_probe
/5HEX/TTCAGCTATGCCGATCTGCGCGATGCCAGT/idSp/
TCAAGGCCTGCCGTC/3IABkFQ/,
o) gyrA_quench_probe
/5Cy3/TCTAGCGATCGGGTCGGTTTACGCATCGTC/idSp/
TCGAGATCAAGCGCG/3IABkRQ/,
p) gyrB_quench_probe
/5Cy5/TGCAGTGGAACGACAGCTACAACGAGAACG/idSp/
GCTGTGCTTCACCAAC/3IABkRQ/,
q) parE_quench_probe
/56-
FAM/TCTTGAAAAATGTGACCTTGTCCTTGACGG/idSp/
CAAGCGAACAGATGA/3IABkFQ/,
r) parC_quench_probe
/5HEX/TGAAATGCACGGTAATAACGGTTCTATGGA/idSp/
GGAGATCCTCCTGCG/3IABkFQ/,
s) rpoB_quench_probe
/5Cy3/TGCGCATCGACCGCAAACGCCGGCAACCGG/idSp/
CACCGTGCTGCTCAAG/3IABkRQ/,
t) cfr_quench_probe
/5Cy5/TCATCACAATGCGGATGTAATTTTGGGTGT/idSp/
AATTTTGTGCTACAG/3IABkRQ/,
and
u) MefA_quench_probe
/56-FAM/CAGGCTATAGTCAGTCTTTGCAGTCTATAAGC/idSp/
ATATTGTTAGTCCGGC/3IABkFQ/.

9. The system of claim 6, wherein the pathogen and/or microbial resistance is detected using a multiplex assay comprising at least two sets of primers and at least two probes.

10. The system of claim 9, wherein the multiplex assay detects both mecA and mef(A).

11. The system of claim 1, wherein the system detects mef(A).

12. The system of claim 1, wherein the sample comprises a bodily fluid.

13. The system of claim 12, wherein the bodily fluid is selected from blood, or a component thereof, urine, or saliva.

14. The system of claim 4, wherein the nuclease is nfo.
